SEQUENCE LISTING (136 sequences)

SEQ ID NO: 1 - DNA, 45 bp, synthetic construct
  source 1..45: mol_type = other DNA
tccgtcatcgcgcttctgggcatcaccatgatttacacggtgaca

SEQ ID NO: 2 - DNA, 45 bp, synthetic construct
  source 1..45: mol_type = other DNA
atggcagtggccgtgttcctctggggcttctatgcctaagaccaa

SEQ ID NO: 3 - DNA, 45 bp, synthetic construct
  source 1..45: mol_type = other DNA
gaggtgggcgggtattcgggtctcttcgacaaatacctgggagca

SEQ ID NO: 4 - DNA, 45 bp, synthetic construct
  source 1..45: mol_type = other DNA
gttggggcctctctcttcgccagcaacatcggcagtggccacttt

SEQ ID NO: 5 - DNA, 45 bp, synthetic construct
  source 1..45: mol_type = other DNA
ccgagctgggcccgtgtggtcaacctcaatgccctgctcatgatg

SEQ ID NO: 6 - DNA, 45 bp, synthetic construct
  source 1..45: mol_type = other DNA
tgaagctcatgcccaacggtctgcgcggactcatgctggcggtca

SEQ ID NO: 7 - DNA, 45 bp, synthetic construct
  source 1..45: mol_type = other DNA
gatggcagtggccgtgttcctctggggcttctatgcctaagacca

SEQ ID NO: 8 - DNA, 45 bp, synthetic construct
  source 1..45: mol_type = other DNA
ggcagtggccactttgtgggcctggcagggactggcgctgcaagt

SEQ ID NO: 9 - DNA, 45 bp, synthetic construct
  source 1..45: mol_type = other DNA
ccgtcatcgcgcttctgggcatcaccatgatttacacggtgacag

SEQ ID NO: 10 - DNA, 45 bp, synthetic construct
  source 1..45: mol_type = other DNA
gctgttgctggattcgagtggaatgcgctcttcgtggtgctgcta

SEQ ID NO: 11 - DNA, 45 bp, synthetic construct
  source 1..45: mol_type = other DNA
gcatagcaaggaggaacgggaggacctggatgctgatgagcagca

SEQ ID NO: 12 - DNA, 45 bp, synthetic construct
  source 1..45: mol_type = other DNA
attcccgagttctccttcggctcgggcagctgtgtgcagccctcg

SEQ ID NO: 13 - DNA, 45 bp, synthetic construct
  source 1..45: mol_type = other DNA
gctcatgcccaacggtctgcgcggactcatgctggcggtcatgct

SEQ ID NO: 14 - DNA, 45 bp, synthetic construct
  source 1..45: mol_type = other DNA
cgagctgggcccgtgtggtcaacctcaatgccctgctcatgatgg

SEQ ID NO: 15 - DNA, 45 bp, synthetic construct
  source 1..45: mol_type = other DNA
tcatgcccaacggtctgcgcggactcatgctggcggtcatgctgg

SEQ ID NO: 16 - DNA, 45 bp, synthetic construct
  source 1..45: mol_type = other DNA
catagcaaggaggaacgggaggacctggatgctgatgagcagcaa

SEQ ID NO: 17 - DNA, 45 bp, synthetic construct
  source 1..45: mol_type = other DNA
aagctgacgcccatgtttctcatggtcatgccaggcatgatcagc

SEQ ID NO: 18 - DNA, 45 bp, synthetic construct
  source 1..45: mol_type = other DNA
gcaggcggcacagggcgggcagctcttcgattacatccaggcagt

SEQ ID NO: 19 - DNA, 45 bp, synthetic construct
  source 1..45: mol_type = other DNA
ctccgtcatcgcgcttctgggcatcaccatgatttacacggtgac

SEQ ID NO: 20 - DNA, 45 bp, synthetic construct
  source 1..45: mol_type = other DNA
aacagaggcactgtgggcggctacttcctggcaggacgcagcatg

SEQ ID NO: 21 - DNA, 45 bp, synthetic construct
  source 1..45: mol_type = other DNA
ctggattcgagtggaatgcgctcttcgtggtgctgctactgggct

SEQ ID NO: 22 - DNA, 45 bp, synthetic construct
  source 1..45: mol_type = other DNA
tgttgctggattcgagtggaatgcgctcttcgtggtgctgctact

SEQ ID NO: 23 - DNA, 45 bp, synthetic construct
  source 1..45: mol_type = other DNA
gcagtggccactttgtgggcctggcagggactggcgctgcaagtg

SEQ ID NO: 24 - DNA, 45 bp, synthetic construct
  source 1..45: mol_type = other DNA
ctcatgatggcagtggccgtgttcctctggggcttctatgcctaa

SEQ ID NO: 25 - RNA, 20 nt, synthetic construct
  source 1..20: mol_type = other RNA
taaatcatggtgatgcccag

SEQ ID NO: 26 - RNA, 20 nt, synthetic construct
  source 1..20: mol_type = other RNA
gcatagaagccccagaggaa

SEQ ID NO: 27 - RNA, 20 nt, synthetic construct
  source 1..20: mol_type = other RNA
tatttgtcgaagagacccga

SEQ ID NO: 28 - RNA, 20 nt, synthetic construct
  source 1..20: mol_type = other RNA
ctgccgatgttgctggcgaa

SEQ ID NO: 29 - RNA, 20 nt, synthetic construct
  source 1..20: mol_type = other RNA
agggcattgaggttgaccac

SEQ ID NO: 30 - RNA, 20 nt, synthetic construct
  source 1..20: mol_type = other RNA
catgagtccgcgcagaccgt

SEQ ID NO: 31 - RNA, 20 nt, synthetic construct
  source 1..20: mol_type = other RNA
catagaagccccagaggaac

SEQ ID NO: 32 - RNA, 20 nt, synthetic construct
  source 1..20: mol_type = other RNA
ccagtccctgccaggcccac

SEQ ID NO: 33 - RNA, 20 nt, synthetic construct
  source 1..20: mol_type = other RNA
gtaaatcatggtgatgccca

SEQ ID NO: 34 - RNA, 20 nt, synthetic construct
  source 1..20: mol_type = other RNA
acgaagagcgcattccactc

SEQ ID NO: 35 - RNA, 20 nt, synthetic construct
  source 1..20: mol_type = other RNA
cagcatccaggtcctcccgt

SEQ ID NO: 36 - RNA, 20 nt, synthetic construct
  source 1..20: mol_type = other RNA
acacagctgcccgagccgaa

SEQ ID NO: 37 - RNA, 20 nt, synthetic construct
  source 1..20: mol_type = other RNA
ccagcatgagtccgcgcaga

SEQ ID NO: 38 - RNA, 20 nt, synthetic construct
  source 1..20: mol_type = other RNA
cagggcattgaggttgacca

SEQ ID NO: 39 - RNA, 20 nt, synthetic construct
  source 1..20: mol_type = other RNA
cgccagcatgagtccgcgca

SEQ ID NO: 40 - RNA, 20 nt, synthetic construct
  source 1..20: mol_type = other RNA
tcagcatccaggtcctcccg

SEQ ID NO: 41 - RNA, 20 nt, synthetic construct
  source 1..20: mol_type = other RNA
cctggcatgaccatgagaaa

SEQ ID NO: 42 - RNA, 20 nt, synthetic construct
  source 1..20: mol_type = other RNA
tgtaatcgaagagctgcccg

SEQ ID NO: 43 - RNA, 20 nt, synthetic construct
  source 1..20: mol_type = other RNA
aaatcatggtgatgcccaga

SEQ ID NO: 44 - RNA, 20 nt, synthetic construct
  source 1..20: mol_type = other RNA
cctgccaggaagtagccgcc

SEQ ID NO: 45 - RNA, 20 nt, synthetic construct
  source 1..20: mol_type = other RNA
cagcaccacgaagagcgcat

SEQ ID NO: 46 - RNA, 20 nt, synthetic construct
  source 1..20: mol_type = other RNA
ccacgaagagcgcattccac

SEQ ID NO: 47 - RNA, 20 nt, synthetic construct
  source 1..20: mol_type = other RNA
gccagtccctgccaggccca

SEQ ID NO: 48 - RNA, 20 nt, synthetic construct
  source 1..20: mol_type = other RNA
aagccccagaggaacacggc

SEQ ID NO: 49 - RNA, 21 nt, synthetic construct
  source 1..21: mol_type = other RNA
taaatcatggtgatgcccatt

SEQ ID NO: 50 - RNA, 21 nt, synthetic construct
  source 1..21: mol_type = other RNA
tcatagaagccccagaggatt

SEQ ID NO: 51 - RNA, 21 nt, synthetic construct
  source 1..21: mol_type = other RNA
tatttgtcgaagagacccgtt

SEQ ID NO: 52 - RNA, 21 nt, synthetic construct
  source 1..21: mol_type = other RNA
ttgccgatgttgctggcgatt

SEQ ID NO: 53 - RNA, 21 nt, synthetic construct
  source 1..21: mol_type = other RNA
tgggcattgaggttgaccatt

SEQ ID NO: 54 - RNA, 21 nt, synthetic construct
  source 1..21: mol_type = other RNA
tatgagtccgcgcagaccgtt

SEQ ID NO: 55 - RNA, 21 nt, synthetic construct
  source 1..21: mol_type = other RNA
tatagaagccccagaggaatt

SEQ ID NO: 56 - RNA, 21 nt, synthetic construct
  source 1..21: mol_type = other RNA
tcagtccctgccaggcccatt

SEQ ID NO: 57 - RNA, 21 nt, synthetic construct
  source 1..21: mol_type = other RNA
ttaaatcatggtgatgccctt

SEQ ID NO: 58 - RNA, 21 nt, synthetic construct
  source 1..21: mol_type = other RNA
tcgaagagcgcattccacttt

SEQ ID NO: 59 - RNA, 21 nt, synthetic construct
  source 1..21: mol_type = other RNA
tagcatccaggtcctcccgtt

SEQ ID NO: 60 - RNA, 21 nt, synthetic construct
  source 1..21: mol_type = other RNA
tcacagctgcccgagccgatt

SEQ ID NO: 61 - RNA, 21 nt, synthetic construct
  source 1..21: mol_type = other RNA
tcagcatgagtccgcgcagtt

SEQ ID NO: 62 - RNA, 21 nt, synthetic construct
  source 1..21: mol_type = other RNA
tagggcattgaggttgacctt

SEQ ID NO: 63 - RNA, 21 nt, synthetic construct
  source 1..21: mol_type = other RNA
tgccagcatgagtccgcgctt

SEQ ID NO: 64 - RNA, 21 nt, synthetic construct
  source 1..21: mol_type = other RNA
tcagcatccaggtcctccctt

SEQ ID NO: 65 - RNA, 21 nt, synthetic construct
  source 1..21: mol_type = other RNA
tctggcatgaccatgagaatt

SEQ ID NO: 66 - RNA, 21 nt, synthetic construct
  source 1..21: mol_type = other RNA
tgtaatcgaagagctgccctt

SEQ ID NO: 67 - RNA, 21 nt, synthetic construct
  source 1..21: mol_type = other RNA
taatcatggtgatgcccagtt

SEQ ID NO: 68 - RNA, 21 nt, synthetic construct
  source 1..21: mol_type = other RNA
tctgccaggaagtagccgctt

SEQ ID NO: 69 - RNA, 21 nt, synthetic construct
  source 1..21: mol_type = other RNA
tagcaccacgaagagcgcatt

SEQ ID NO: 70 - RNA, 21 nt, synthetic construct
  source 1..21: mol_type = other RNA
tcacgaagagcgcattccatt

SEQ ID NO: 71 - RNA, 21 nt, synthetic construct
  source 1..21: mol_type = other RNA
tccagtccctgccaggccctt

SEQ ID NO: 72 - RNA, 21 nt, synthetic construct
  source 1..21: mol_type = other RNA
tagccccagaggaacacggtt

SEQ ID NO: 73 - RNA, 16 nt, synthetic construct
  source 1..16: mol_type = other RNA
gcatcaccatgattta

SEQ ID NO: 74 - RNA, 16 nt, synthetic construct
  source 1..16: mol_type = other RNA
tctggggcttctatga

SEQ ID NO: 75 - RNA, 16 nt, synthetic construct
  source 1..16: mol_type = other RNA
gtctcttcgacaaata

SEQ ID NO: 76 - RNA, 16 nt, synthetic construct
  source 1..16: mol_type = other RNA
ccagcaacatcggcaa

SEQ ID NO: 77 - RNA, 16 nt, synthetic construct
  source 1..16: mol_type = other RNA
tcaacctcaatgccca

SEQ ID NO: 78 - RNA, 16 nt, synthetic construct
  source 1..16: mol_type = other RNA
tctgcgcggactcata

SEQ ID NO: 79 - RNA, 16 nt, synthetic construct
  source 1..16: mol_type = other RNA
ctctggggcttctata

SEQ ID NO: 80 - RNA, 16 nt, synthetic construct
  source 1..16: mol_type = other RNA
gcctggcagggactga

SEQ ID NO: 81 - RNA, 16 nt, synthetic construct
  source 1..16: mol_type = other RNA
catcaccatgatttaa

SEQ ID NO: 82 - RNA, 16 nt, synthetic construct
  source 1..16: mol_type = other RNA
ggaatgcgctcttcga

SEQ ID NO: 83 - RNA, 16 nt, synthetic construct
  source 1..16: mol_type = other RNA
gaggacctggatgcta

SEQ ID NO: 84 - RNA, 16 nt, synthetic construct
  source 1..16: mol_type = other RNA
gctcgggcagctgtga

SEQ ID NO: 85 - RNA, 16 nt, synthetic construct
  source 1..16: mol_type = other RNA
cgcggactcatgctga

SEQ ID NO: 86 - RNA, 16 nt, synthetic construct
  source 1..16: mol_type = other RNA
caacctcaatgcccta

SEQ ID NO: 87 - RNA, 16 nt, synthetic construct
  source 1..16: mol_type = other RNA
cggactcatgctggca

SEQ ID NO: 88 - RNA, 16 nt, synthetic construct
  source 1..16: mol_type = other RNA
aggacctggatgctga

SEQ ID NO: 89 - RNA, 16 nt, synthetic construct
  source 1..16: mol_type = other RNA
tcatggtcatgccaga

SEQ ID NO: 90 - RNA, 16 nt, synthetic construct
  source 1..16: mol_type = other RNA
cagctcttcgattaca

SEQ ID NO: 91 - RNA, 16 nt, synthetic construct
  source 1..16: mol_type = other RNA
ggcatcaccatgatta

SEQ ID NO: 92 - RNA, 16 nt, synthetic construct
  source 1..16: mol_type = other RNA
gctacttcctggcaga

SEQ ID NO: 93 - RNA, 16 nt, synthetic construct
  source 1..16: mol_type = other RNA
gctcttcgtggtgcta

SEQ ID NO: 94 - RNA, 16 nt, synthetic construct
  source 1..16: mol_type = other RNA
aatgcgctcttcgtga

SEQ ID NO: 95 - RNA, 16 nt, synthetic construct
  source 1..16: mol_type = other RNA
cctggcagggactgga

SEQ ID NO: 96 - RNA, 16 nt, synthetic construct
  source 1..16: mol_type = other RNA
tgttcctctggggcta

SEQ ID NO: 97 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
  modified_base 20..21: mod_base = OTHER; note = Extended nucleic acid-modified internucleotide linkage
ttaatctctttactgatattt

SEQ ID NO: 98 - RNA, 16 nt, synthetic construct
  source 1..16: mol_type = other RNA
  modified_base order(1..6,8..10,12,14,16): mod_base = OTHER; note = 2-Prime-O-methyl nucleotides
  modified_base order(7,11,13,15): mod_base = OTHER; note = 2-Prime-fluoro nucleotides
  modified_base order(1..3,14..16): mod_base = OTHER; note = Phosphorothioate internucleotide linkage
atatcagtaaagagat

SEQ ID NO: 99 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,17..21): mod_base = OTHER; note = Phosphorothioate internucleotide linkage
  modified_base 20..21: mod_base = OTHER; note = Extended nucleic acid-modified internucleotide linkage
ttaatctctttactgatattt

SEQ ID NO: 100 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,19..21): mod_base = OTHER; note = Phosphorothioate internucleotide linkage
  modified_base 20..21: mod_base = OTHER; note = Extended nucleic acid-modified internucleotide linkage
ttaatctctttactgatattt

SEQ ID NO: 101 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,17..21): mod_base = OTHER; note = Phosphorothioate internucleotide linkage
  modified_base 20..21: mod_base = OTHER; note = Extended nucleic acid-modified internucleotide linkage
tatcgggaagctttgtcagtt

SEQ ID NO: 102 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,17..21): mod_base = OTHER; note = Phosphorothioate internucleotide linkage
ttaatctctttactgatattt

SEQ ID NO: 103 - RNA, 21 nt, synthetic construct
  source 1..21: mol_type = other RNA
  modified_base order(1,3,7,9,11,13,15,17..19): mod_base = OTHER; note = 2-Prime-O-methyl nucleotides
  modified_base order(2,4..6,8,10,12,14,16): mod_base = OTHER; note = 2-Prime-fluoro nucleotides
  modified_base order(1..3,17..21): mod_base = OTHER; note = Phosphorothioate internucleotide linkage
  modified_base 20: mod_base = OTHER; note = 2-Prime-O-methyl-extended nucleic acid
  modified_base 21: mod_base = OTHER; note = 2-Prime-fluoro-extended nucleic acid
ttaatctctttactgatattt

SEQ ID NO: 104 - RNA, 16 nt, synthetic construct
  source 1..16: mol_type = other RNA
  modified_base order(1..3,5..7,9,11,13,15..16): mod_base = OTHER; note = 2-Prime-O-methyl nucleotides
  modified_base order(4,8,10,12,14): mod_base = OTHER; note = 2-Prime-fluoro nucleotides
  modified_base order(1..3,14..16): mod_base = OTHER; note = Phosphorothioate internucleotide linkage
tcagtaaagagattaa

SEQ ID NO: 105 - RNA, 16 nt, synthetic construct
  source 1..16: mol_type = other RNA
  modified_base order(1..3,5..7,9,11,13,15..16): mod_base = OTHER; note = 2-Prime-O-methyl nucleotides
  modified_base order(4,8,10,12,14): mod_base = OTHER; note = 2-Prime-fluoro nucleotides
  modified_base order(1..3,14..16): mod_base = OTHER; note = Phosphorothioate internucleotide linkage
tcagtaaagagattaa

SEQ ID NO: 106 - RNA, 16 nt, synthetic construct
  source 1..16: mol_type = other RNA
  modified_base order(1..3,5..7,9,11,13,15..16): mod_base = OTHER; note = 2-Prime-O-methyl nucleotides
  modified_base order(4,8,10,12,14): mod_base = OTHER; note = 2-Prime-fluoro nucleotides
  modified_base order(1..3,14..16): mod_base = OTHER; note = Phosphorothioate internucleotide linkage
tcagtaaagagattaa

SEQ ID NO: 107 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
taaatcatggtgatgcccatt

SEQ ID NO: 108 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tcatagaagccccagaggatt

SEQ ID NO: 109 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tatttgtcgaagagacccgtt

SEQ ID NO: 110 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
ttgccgatgttgctggcgatt

SEQ ID NO: 111 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tgggcattgaggttgaccatt

SEQ ID NO: 112 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tatgagtccgcgcagaccgtt

SEQ ID NO: 113 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tatagaagccccagaggaatt

SEQ ID NO: 114 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tcagtccctgccaggcccatt

SEQ ID NO: 115 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
ttaaatcatggtgatgccctt

SEQ ID NO: 116 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tcgaagagcgcattccacttt

SEQ ID NO: 117 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tagcatccaggtcctcccgtt

SEQ ID NO: 118 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tcacagctgcccgagccgatt

SEQ ID NO: 119 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tcagcatgagtccgcgcagtt

SEQ ID NO: 120 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tagggcattgaggttgacctt

SEQ ID NO: 121 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tgccagcatgagtccgcgctt

SEQ ID NO: 122 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tcagcatccaggtcctccctt

SEQ ID NO: 123 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tctggcatgaccatgagaatt

SEQ ID NO: 124 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tgtaatcgaagagctgccctt

SEQ ID NO: 125 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
taatcatggtgatgcccagtt

SEQ ID NO: 126 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tctgccaggaagtagccgctt

SEQ ID NO: 127 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tagcaccacgaagagcgcatt

SEQ ID NO: 128 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tcacgaagagcgcattccatt

SEQ ID NO: 129 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tccagtccctgccaggccctt

SEQ ID NO: 130 - RNA, 21 nt, synthetic construct
  source 1..21: mol_type = other RNA
  modified_base order(1,3,7,9,11,13,15,17..20): mod_base = OTHER; note = 2-Prime-O-methyl nucleotides
  modified_base order(2,4..6,8,10,12,14,16,21): mod_base = OTHER; note = 2-Prime-fluoro nucleotides
  modified_base order(1..3,14..21): mod_base = OTHER; note = Phosphorothioate internucleotide linkage
tagccccagaggaacacggtt

SEQ ID NO: 131 - RNA, 21 nt, synthetic construct
  source 1..21: mol_type = other RNA
  modified_base order(1,3,7,9,11,13,15,17..19): mod_base = OTHER; note = 2-Prime-O-methyl nucleotides
  modified_base order(2,4..6,8,10,12,14,16): mod_base = OTHER; note = 2-Prime-fluoro nucleotides
  modified_base order(1..3,17..21): mod_base = OTHER; note = Phosphorothioate internucleotide linkage
  modified_base 20: mod_base = OTHER; note = 2-Prime-O-methyl-extended nucleic acid
  modified_base 21: mod_base = OTHER; note = 2-Prime-fluoro-extended nucleic acid
tcacgaagagcgcattccatt

SEQ ID NO: 132 - RNA, 21 nt, synthetic construct
  source 1..21: mol_type = other RNA
  modified_base order(1,3,7,9,11,13,15,17..19): mod_base = OTHER; note = 2-Prime-O-methyl nucleotides
  modified_base order(2,4..6,8,10,12,14,16): mod_base = OTHER; note = 2-Prime-fluoro nucleotides
  modified_base order(1..3,17..21): mod_base = OTHER; note = Phosphorothioate internucleotide linkage
  modified_base 20: mod_base = OTHER; note = 2-Prime-O-methyl-extended nucleic acid
  modified_base 21: mod_base = OTHER; note = 2-Prime-fluoro-extended nucleic acid
taaatcatggtgatgcccatt

SEQ ID NO: 133 - RNA, 16 nt, synthetic construct
  source 1..16: mol_type = other RNA
  modified_base order(1..3,5,7,9,11..13,15..16): mod_base = OTHER; note = 2-Prime-O-methyl nucleotides
  modified_base order(4,6,8,10,14): mod_base = OTHER; note = 2-Prime-fluoro nucleotides
  modified_base order(1..3,14..16): mod_base = OTHER; note = Phosphorothioate internucleotide linkage
aatgcgctcttcgtga

SEQ ID NO: 134 - RNA, 16 nt, synthetic construct
  source 1..16: mol_type = other RNA
  modified_base order(1..3,5,7,9,11..13,15..16): mod_base = OTHER; note = 2-Prime-O-methyl nucleotides
  modified_base order(4,6,8,10,14): mod_base = OTHER; note = 2-Prime-fluoro nucleotides
  modified_base order(1..3,14..16): mod_base = OTHER; note = Phosphorothioate internucleotide linkage
gcatcaccatgattta

SEQ ID NO: 135 - RNA, 16 nt, synthetic construct
  source 1..16: mol_type = other RNA
  modified_base order(1..3,5,7,9,11..13,15..16): mod_base = OTHER; note = 2-Prime-O-methyl nucleotides
  modified_base order(4,6,8,10,14): mod_base = OTHER; note = 2-Prime-fluoro nucleotides
  modified_base order(1..3,14..16): mod_base = OTHER; note = Phosphorothioate internucleotide linkage
aatgcgctcttcgtga

SEQ ID NO: 136 - RNA, 16 nt, synthetic construct
  source 1..16: mol_type = other RNA
  modified_base order(1..3,5,7,9,11..13,15..16): mod_base = OTHER; note = 2-Prime-O-methyl nucleotides
  modified_base order(4,6,8,10,14): mod_base = OTHER; note = 2-Prime-fluoro nucleotides
  modified_base order(1..3,14..16): mod_base = OTHER; note = Phosphorothioate internucleotide linkage
gcatcaccatgattta

(19) United States
(12) Patent Application Publication
(10) Pub. No.: US 2025/0066780 A1
(43) Pub. Date: Feb. 27, 2025

(54) OLIGONUCLEOTIDES WITH INTERNUCLEOTIDE LINKAGE MODIFICATIONS FOR KIDNEY TARGETING

(71) Applicant: UNIVERSITY OF MASSACHUSETTS, Boston, MA (US)

(72) Inventors: Anastasia Khvorova, Westborough, MA (US); Ken Yamada, Westborough, MA (US); Vignesh Narayan Hariharan, Natick, MA (US); Julianna Buchwald, Boston, MA (US)

(21) Appl. No.: 18/756,700

(22) Filed: Jun. 27, 2024

Related U.S. Application Data
(60) Provisional application No. 63/523,830, filed on Jun. 28, 2023.

(51) Int. Cl. C12N 15/113

(52) U.S. Cl. CPC C12N 15/113; C12N 2310/11; C12N 2310/14; C12N 2310/315; C12N 2320/32

(57) ABSTRACT

This disclosure relates to oligonucleotides with internucleotide linkage modifications and used thereof for treating and preventing kidney diseases. In particular, the present disclosure provides compositions, systems, and methods for the delivery of therapeutic oligonucleotide to kidney. The oligonucleotide disclosed herein can be delivered to the kidney upon administration.

### CROSS REFERENCE TO RELATED APPLICATION

This application claims the benefit of U.S. Provisional Patent Application Ser. No. 63/523,830, filed Jun. 28, 2023. The entire content of the above-referenced patent application is incorporated by reference in its entirety herein.

### SEQUENCE LISTING

The instant application contains a Sequence Listing which has been submitted electronically in XML format and is hereby incorporated by reference in its entirety. Said XML file, created on Nov. 13, 2024, is named 754856_UM9-306_ST26.xml and is 196,680 bytes in size.

### FIELD OF THE DISCLOSURE

This disclosure relates to oligonucleotides with internucleotide linkage modifications and used thereof for treating and preventing kidney diseases.

### BACKGROUND

RNA therapeutics is changing the world of medicine, where the development of efficient tissue-specific delivery is a foundation for successful clinical translation. Kidney is a highly metabolically active tissue with many of targets critical for many diseases. Robust kidney gene modulation represents a primary unmet technology need, where silencing in many regions, including proximal epithelial, glomerulus, and bowman capsule, are of clinical interest. While uptake in the kidney can be achieved, it is mainly driven by non-specific entrapment of oligonucleotides during filtration and thus is primarily non-functionally productive, which resulted in non-efficacious target gene silencing. Therefore, there remains a need for improved kidney disease prevention and treatment.

### SUMMARY

The present disclosure provides oligonucleotides with internucleotide linkage modifications and used thereof for treating and preventing kidney diseases.

In one aspect, the disclosure provides a method for delivering an oligonucleotide to the kidney of a subject, the method comprising administering to the subject an effective amount of the oligonucleotide, wherein the oligonucleotide comprises a 5′ end, a 3′ end, at least one exNA intersubunit linkage, and complementarity to a target nucleic acid, and wherein the oligonucleotide is formulated for kidney administration.

In some embodiments, the oligonucleotide comprises the at least one exNA intersubunit linkage between one or more of the positions 1 to 4, 1 to 3, and 1 to 2 from the 3′ end of the oligonucleotide.

In some embodiments, the oligonucleotide comprises the at least one exNA intersubunit linkage between the positions 1 to 2 and 2 to 3 from the 3′ end of the oligonucleotide.

In some embodiments, the nucleotide at one or more of the positions 1, 2, 3, and 4 is U.

In some embodiments, the nucleotide at positions 1 and 2 is U.

In some embodiments, the at least one exNA intersubunit linkage comprises the intersubunit linkage of Formula Ia:

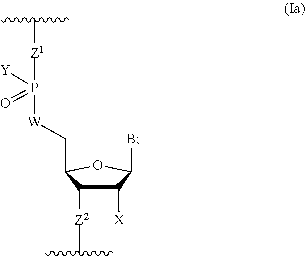

wherein:
B is a base moiety;
W is O or O(CH₂)ₙ¹, wherein n¹ is 1 to 10;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z¹ is O or O(CH₂)ₙ², wherein n² is 1 to 10;
Z² is O or O(CH₂)ₙ³, wherein n³ is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof, wherein:
Z₁ is O(CH₂)ₙ² and W is O, or Z₁ is O and W is O(CH₂)ₙ¹, or Z₁ is O(CH₂)ₙ² and W is O(CH₂)ₙ¹.

In some embodiments, the Z¹ is O(CH₂)ₙ², n² is 1, W is O, and Y is O⁻.

In some embodiments, the Z¹ is O, W is O(CH₂)ₙ¹, n¹ is 1, and Y is O⁻.

In some embodiments, the Z¹ is O(CH₂)ₙ², n² is 1, W is O, and Y is S⁻.

In some embodiments, the Z¹ is O(CH₂)ₙ², n² is 1, W is O(CH₂)ₙ¹, and Y is O⁻.

In some embodiments, the Z¹ is O(CH₂)ₙ², n² is 1, W is O(CH₂)ₙ¹, and Y is S⁻.

In some embodiments, the Y is S⁻.

In some embodiments, the X is OR¹ or F.

In some embodiments, the base moiety is selected from the group consisting of adenine, guanine, cytosine, and uracil.

In some embodiments, the oligonucleotide corresponds to an antisense oligonucleotide or a siRNA.

In some embodiments, the siRNA comprises a sense strand and an antisense strand.

In some embodiments, the oligonucleotide comprises the structure:

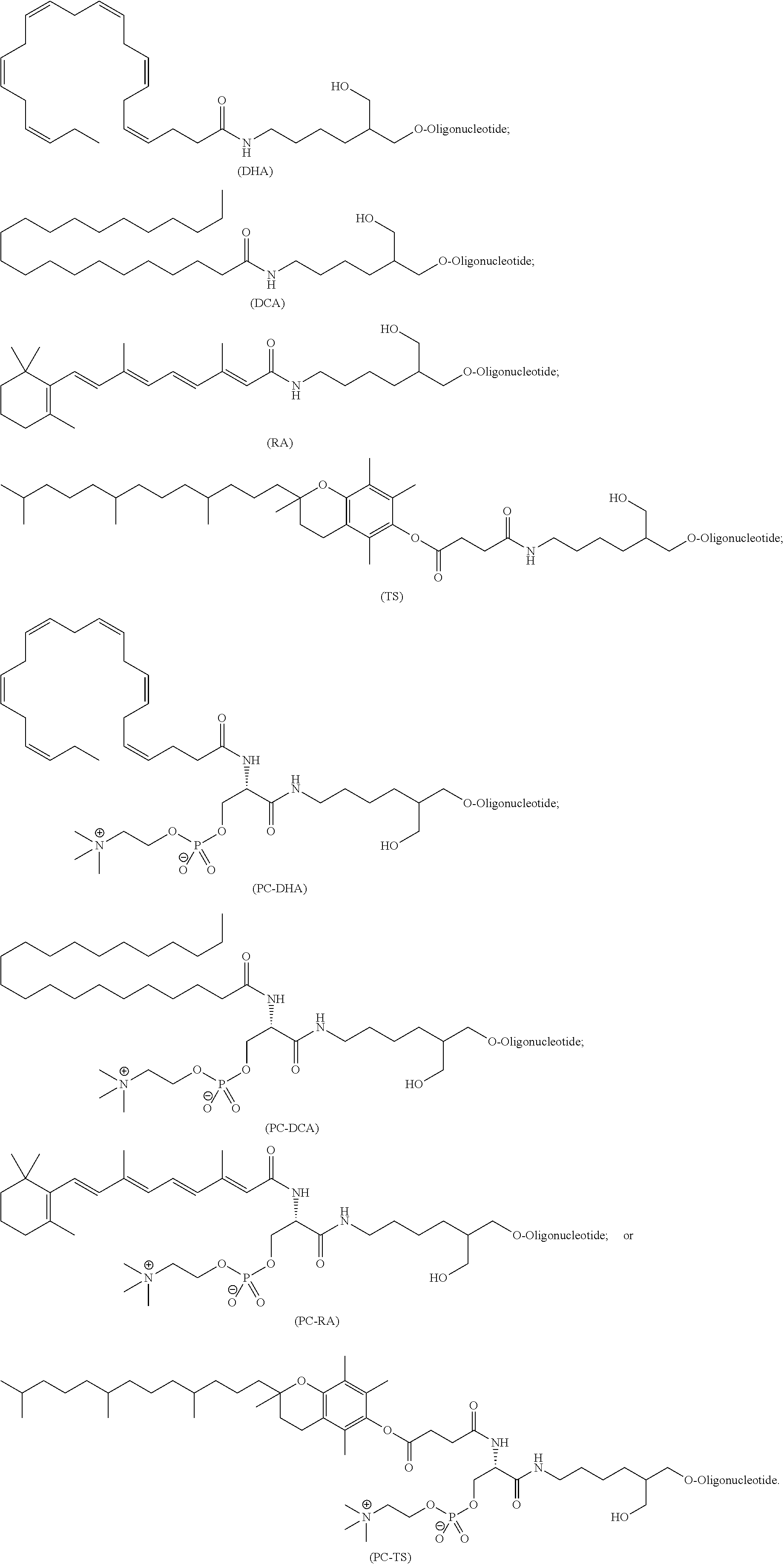

In some embodiments, the oligonucleotide comprises the structure:

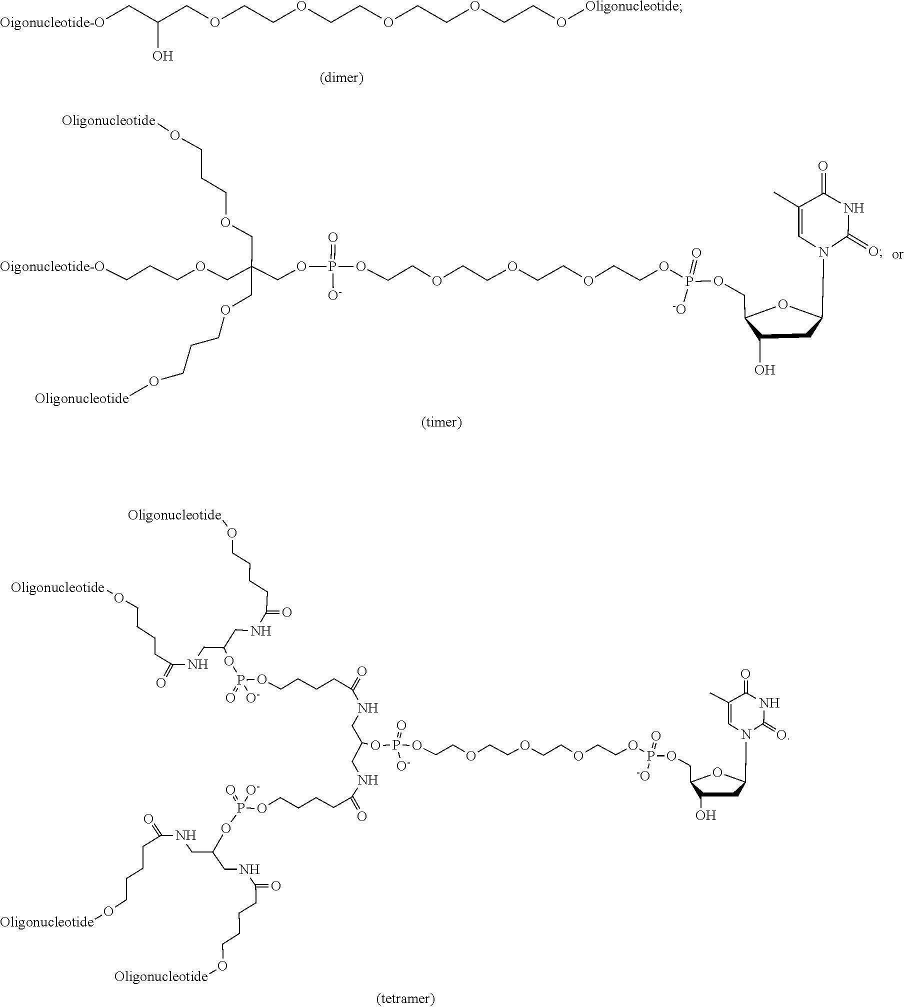

In some embodiments, the antisense strand comprises about 15 nucleotides to about 25 nucleotides in length.

In some embodiments, the sense strand comprises about 15 nucleotides to about 25 nucleotides in length.

In some embodiments, the antisense strand is 20 nucleotides in length, 21 nucleotides in length, or 22 nucleotides in length.

In some embodiments, the sense strand is 15 nucleotides in length, 16 nucleotides in length, 18 nucleotides in length, 19 nucleotides in length, or 20 nucleotides in length.

In some embodiments, the siRNA comprises a double-stranded region of 15 base pairs to 20 base pairs.

In some embodiments, the siRNA comprises a double-stranded region of 15 base pairs, 16 base pairs, 18 base pairs, or 20 base pairs.

In some embodiments, the siRNA comprises at least one blunt-end.

In some embodiments, the siRNA comprises at least one single-stranded nucleotide overhang.

In some embodiments, the siRNA comprises naturally occurring nucleotides.

In some embodiments, the siRNA comprises at least one modified nucleotide.

In some embodiments, the at least one modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In some embodiments, the siRNA comprises at least one modified internucleotide linkage.

In some embodiments, the at least one modified internucleotide linkage comprises a phosphorothioate internucleotide linkage.

In some embodiments, the siRNA comprises 4-16 phosphorothioate internucleotide linkages.

In some embodiments, the siRNA comprises 8-13 phosphorothioate internucleotide linkages.

In some embodiments, the siRNA comprises at least 80% chemically modified nucleotides.

In some embodiments, the siRNA is fully chemically modified.

In some embodiments, the sense strand comprises one or more nucleotide mismatches between the antisense strand and the sense strand.

In some embodiments, the antisense strand comprises a 5′ phosphate, a 5′-alkyl phosphonate, a 5′ alkylene phosphonate, or a 5′ alkenyl phosphonate.

In some embodiments, the antisense strand comprises a 5′ vinyl phosphonate.

In some embodiments, a functional moiety is linked to the 5′ end and/or the 3′ end of the antisense strand.

In some embodiments, a functional moiety is linked to the 5′ end and/or the 3′ end of the sense strand.

In some embodiments, a functional moiety is linked to the 3′ end of the sense strand.

In some embodiments, the functional moiety comprises an N-acetylgalactosamine (GalNAc) moiety.

In some embodiments, the functional moiety comprises a hydrophobic moiety.

In some embodiments, the hydrophobic moiety is selected from the group consisting of fatty acids, steroids, secosteroids, lipids, gangliosides, nucleoside analogs, endocannabinoids, vitamins, and a mixture thereof.

In some embodiments, the steroid is selected from the group consisting of cholesterol and lithocholic acid (LCA).

In some embodiments, the fatty acid is selected from the group consisting of eicosapentaenoic acid (EPA), docosahexaenoic acid (DHA) and docosanoic acid (DCA).

In some embodiments, the functional moiety comprises a lipophilic moiety.

In some embodiments, the lipophilic moiety is selected from the group consisting of cholesterols, vitamin E, vitamin K, vitamin A, folic acids, cationic dyes, and a mixture thereof.

In some embodiments, the lipophilic moiety is selected from the group consisting of cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, phenoxazine, and a mixture thereof.

In some embodiments, the functional moiety is linked to the antisense strand and/or the sense strand by a linker.

In some embodiments, the linker is a cleavable linker, optionally wherein the cleavable linker comprises a phosphodiester linkage, a disulfide linkage, an acid-labile linkage, a photocleavable linkage, or a dTdT dinucleotide with phosphodiester internucleotide linkages, optionally wherein the acid-labile linkage comprises a β-thiopropionate linkage or a carboxydimethylmaleic anhydride (CDM) linkage.

In some embodiments, the linker comprises a divalent linker or a trivalent linker, optionally wherein the divalent linker or the trivalent linker is selected from the group consisting of:

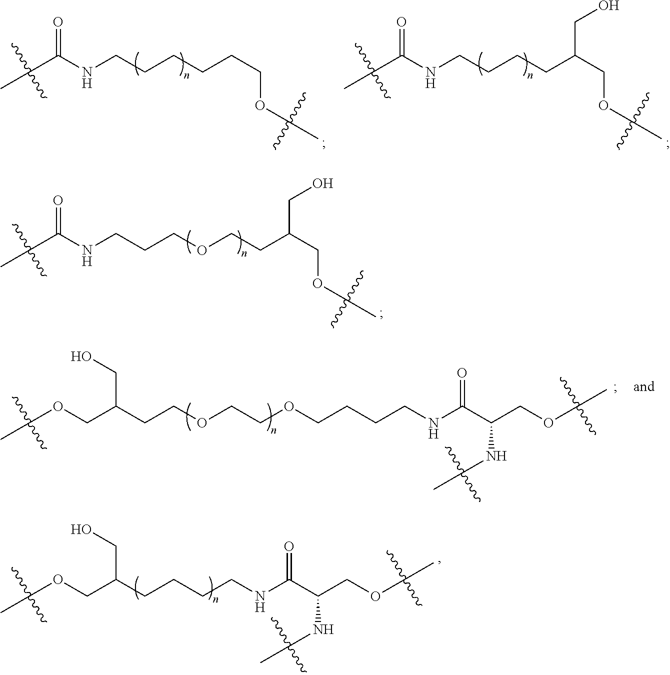

wherein n is 1, 2, 3, 4, or 5.

In some embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In some embodiments, when the linker is a trivalent linker, the linker further links a phosphodiester or phosphodiester derivative.

In some embodiments, the phosphodiester or phosphodiester derivative is selected from the group consisting of:

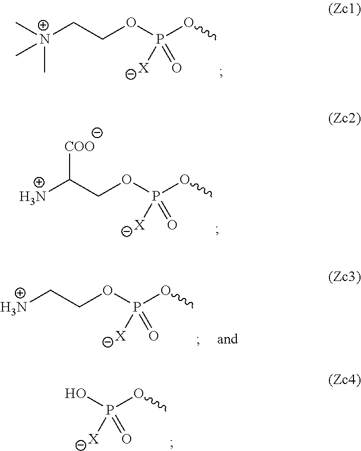

wherein X is O, S, or BH₃.

In some embodiments, the nucleotides at positions 1 and 2 from the 3′ end of sense strand, and the nucleotides at positions 1 and 2 from the 5′ end of antisense strand are connected to adjacent ribonucleotides via phosphorothioate linkages.

In some embodiments, the oligonucleotide is administered to the subject by intravenous (IV) injection, subcutaneous (SQ) injection, or a combination thereof.

In some embodiments, the oligonucleotide formulation comprises about 0.1, 0.5, 1.0, 5.0, 10.0, 15.0, or 20.0 mg/kg of body weight of oligonucleotide.

In some embodiments, the oligonucleotide formulation comprises about 5.0 or about 20.0 mg/kg of body weight of oligonucleotide.

In some embodiments, the oligonucleotide formulation comprises about 5.0 mg/kg of body weight of oligonucleotide.

In some embodiments, the oligonucleotide formulation comprises about 20.0 mg/kg of body weight of oligonucleotide.

In another aspect, the disclosure provides a method for treating a disease, disorder, or injury of the kidney in a patient in need of such treatment, comprising administering to the patient an oligonucleotide comprising a 5′ end, a 3′ end, at least one exNA intersubunit linkage, and complementarity to a target nucleic acid; and wherein the oligonucleotide is formulated for kidney administration.

In some embodiments, the disease, disorder, or injury of the kidney is selected from the group consisting of Chronic Kidney Disease (CKD), Fabry disease, cystinosis, glomerulonephritis, IgA nephropathy, lupus nephritis, atypical hemolytic uremic syndrome (aHUS), Polycystic kidney disease (PKD), and a combination thereof.

In some embodiments, the disease, disorder, or injury of the kidney is associated with a solute carrier family 5 member 2 (SLC5A2) gene.

In some embodiments, the oligonucleotide inhibits the expression of the SLC5A2 gene.

In some embodiments, the target nucleic acid is the sequence of any one of the target region sequences of Table 1.

In another aspect, the disclosure provides a method for administering a therapeutically effective amount of an oligonucleotide to the kidney of a subject, the method comprising administering the oligonucleotide to the subject, wherein the oligonucleotide comprises a 5′ end, a 3′ end, at least one exNA intersubunit linkage, and complementarity to a target nucleic acid; and wherein the oligonucleotide is formulated for kidney administration.

In another aspect, the disclosure provides a method for inhibiting the expression of SLC5A2 gene in a cell, the method comprising:
(a) introducing into the cell an oligonucleotide comprising an oligonucleotide comprising a 5′ end, a 3′ end, at least one exNA intersubunit linkage, and complementarity to a target nucleic acid; and
(b) maintaining the cell produced in step (a) for a time sufficient to obtain degradation of the mRNA transcript of the SLC5A2 gene, thereby inhibiting expression of the SLC5A2 gene in the cell.

In another aspect, the disclosure provides an oligonucleotide for use in the treatment of a disease, disorder, or injury of the kidney in a patient in need of such treatment, wherein the oligonucleotide is administered to the patient and formulated for kidney administration, and wherein the oligonucleotide comprises an oligonucleotide comprising a 5′ end, a 3′ end, at least one exNA intersubunit linkage, and complementarity to a target nucleic acid.

In another aspect, the disclosure provides a pharmaceutical composition for treating a disease, disorder, or injury of the kidney in a patient in need of such treatment, comprising:

An oligonucleotide comprising an oligonucleotide comprising a 5′ end, a 3′ end, at least one exNA intersubunit linkage, and complementarity to a target nucleic acid; and
a pharmaceutically acceptable carrier,
   wherein the pharmaceutical composition is formulated for kidney administration.

These and other aspects of the applicant's teachings are set forth herein.

### BRIEF DESCRIPTION OF THE DRAWINGS

The foregoing and other features and advantages of the present disclosure will be more fully understood from the following detailed description of illustrative embodiments taken in conjunction with the accompanying drawings.

FIG. 1 depicts the identification of hit sequences in vitro. Luminescence measurements from cells transfected with psiCheck-2 vector (Promega) containing siRNA targeting regions cloned upstream of the luciferase reporter gene were performed. Cells were treated with 1.5 uM of siRNA for 72 h following which reporter expression was measured according to manufacturer's instructions. Data represented as mean±s.d. Dotted line represents 50% expression cutoff for hit identification.

FIG. 2 depicts the identification of lead sequences in vitro. Luminescence measurements from cells transfected with psiCheck-2 vector (Promega) containing siRNA targeting regions cloned upstream of the luciferase reporter gene were performed. Cells were treated with increasing concentrations of siRNA for 72 h following which reporter expression was measured according to manufacturer's instructions. Data represented as mean±s.d. IC₅₀ measurements were derived from log(inhibitor) vs. response (four parameters) non-linear fit performed using Graphpad Prism software.

FIG. 3 depicts the identification of lead kidney targeting pattern. Relative huntingtin mRNA expression of FVB mice treated sub-cutaneously with 20 mg/kg siRNA targeting mouse huntingtin (HTT) or non-targeting control (NTC) 2 weeks (left) or 4 weeks (right) post injection in different phosphorothioate (PS) and extended nucleic acid (exNA) scaffold variants. Data represented as mean±s.e.m. of individual animals (n=5 per group). NTC non targeting control, HTT—huntingtin targeting siRNA, PS—phosphorothioate, exNA—extended nucleic acid, *−p<0.05, ***−p<0.001, ****−p<0.0001.

FIG. 4 depicts the tissue specific silencing of siRNA targeting the huntingtin gene. The relative huntingtin mRNA expression in liver, kidney, thymus, and adrenal tissues of FVB mice treated sub-cutaneously with 20 mg/kg siRNA targeting mouse huntingtin (HTT) or non-targeting control (NTC) 2 weeks post injection was assessed. Data represented as mean±s.e.m. of individual animals (n=5 per group). NTC—non targeting control, Unconj—unconjugated, exNA—extended nucleic acid, EPA—eicosapentaenoic acid, and Myr—myristic acid.

FIG. 5 depicts the tissue specific silencing of siRNA targeting the huntingtin gene. The relative huntingtin mRNA expression in heart, muscle, spleen, and lung tissues of FVB mice treated sub-cutaneously with 20 mg/kg siRNA targeting mouse huntingtin (HTT) or non-targeting control (NTC) 2 weeks post injection was assessed. Data represented as mean±s.e.m. of individual animals (n=5 per group). NTC—non targeting control, Unconj—unconjugated, exNA—extended nucleic acid, EPA—eicosapentaenoic acid, and Myr—myristic acid.

FIG. 6 depicts the tissue specific silencing of siRNA targeting the huntingtin gene. The relative huntingtin mRNA expression in fat, stomach, and uterus tissues of FVB mice treated sub-cutaneously with 20 mg/kg siRNA targeting mouse huntingtin (HTT) or non-targeting control (NTC) 2 weeks post injection was assessed. Data represented as mean±s.e.m. of individual animals (n=5 per group). NTC—non targeting control, Unconj—unconjugated, exNA—extended nucleic acid, EPA—eicosapentaenoic acid, and Myr—myristic acid.

FIG. 7 depicts the tissue specific silencing of siRNA targeting the huntingtin gene. The relative huntingtin mRNA expression in liver, kidney, thymus, and adrenal tissues of FVB mice treated sub-cutaneously with 20 mg/kg siRNA targeting mouse huntingtin (HTT) or non-targeting control (NTC) 4 weeks post injection was assessed. Data represented as mean±s.e.m. of individual animals (n=5 per group). NTC—non targeting control, Unconj—unconjugated, exNA—extended nucleic acid, EPA—eicosapentaenoic acid, and Myr—myristic acid.

FIG. 8 depicts the tissue specific silencing of siRNA targeting the huntingtin gene. The relative huntingtin mRNA expression in heart, muscle, spleen, and lung tissues of FVB mice treated sub-cutaneously with 20 mg/kg siRNA targeting mouse huntingtin (HTT) or non-targeting control (NTC) 4 weeks post injection was assessed. Data represented as mean±s.e.m. of individual animals (n=5 per group). NTC—non targeting control, Unconj—unconjugated, exNA—extended nucleic acid, EPA—eicosapentaenoic acid, and Myr—myristic acid.

FIG. 9 depicts the tissue specific silencing of siRNA targeting the huntingtin gene. The relative huntingtin mRNA expression in fat, stomach, and uterus tissues of FVB mice treated sub-cutaneously with 20 mg/kg siRNA targeting mouse huntingtin (HTT) or non-targeting control (NTC) 4 weeks post injection was assessed. Data represented as mean±s.e.m. of individual animals (n=5 per group). NTC—non targeting control, Unconj—unconjugated, exNA—extended nucleic acid, EPA—eicosapentaenoic acid, and Myr—myristic acid.

FIG. 10 depicts the tissue specific accumulation of siRNA targeting the huntingtin gene. The siRNA concentration in kidney, liver, heart and muscle tissues of FVB mice treated sub-cutaneously with 20 mg/kg siRNA targeting mouse huntingtin (HTT) or non-targeting control (NTC) 2- and 4-weeks post injection was assessed. Data represented as mean±s.e.m. of individual animals (n=5 per group). NTC—non targeting control, Unconj—unconjugated, exNA—extended nucleic acid, EPA—eicosapentaenoic acid, and Myr—myristic acid.

FIG. 11 depicts tissue distribution comparisons of siRNA targeting the huntingtin gene. The siRNA concentration in liver, kidney cortex, heart, and muscle tissues of FVB mice treated sub-cutaneously with 20 mg/kg siRNA targeting mouse huntingtin (HTT) or non-targeting control (NTC) 2 weeks post injection was assessed. Data represented as mean±s.e.m. of individual animals (n=5 per group).

FIG. 12 depicts the kidney tissue silencing of siRNA targeting the SLC5A2 gene. The SLC5A2 expression in kidney tissues of FVB mice treated sub-cutaneously with 40 mg/kg siRNA targeting mouse SLC5A2 or non-targeting control (NTC) was assessed. Data represented as mean±s.e.m. of individual animals (n=5 per group). NTC—non targeting control, Unc—unconjugated, and Myr—myristic acid.

FIG. 13 depicts the kidney tissue silencing and the whole blood glucose level in mice injected with siRNA targeting the SLC5A2 gene. The SLC5A2 expression in kidney tissue and glucose level of FVB mice treated sub-cutaneously with 2×20 mg/kg siRNA targeting mouse SLC5A2 or non-targeting control (NTC) 2- and 4-weeks post injection were assessed. Data represented as mean±s.e.m. of individual animals (n=6 per group). NTC—non targeting control.

FIGS. 14A-14B depict whole blood glucose level in mice injected with siRNA targeting the SLC5A2 gene. The whole blood glucose level of C57BL/6 or db/db mice treated sub-cutaneously with 40 mg/kg siRNA targeting mouse SLC5A2 or non-targeting control (NTC) were assessed. FIG. 14A shows data graphed as glucose level in mg/dL and FIG. 14B shows data graphed as % of each animal's baseline. Data represented as mean±s.e.m. of individual animals (n=8 per group). NTC—non targeting control, Myr—myristic acid, and exNA—extended nucleic acid.

### DETAILED DESCRIPTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The present disclosure relates to oligonucleotides with internucleotide linkage modifications and used thereof for treating and preventing kidney diseases. In particular, the present disclosure provides compositions, systems, and methods for the delivery of therapeutic oligonucleotide to kidney. The oligonucleotide disclosed herein can be delivered to the kidney upon administration.

The oligonucleotides described herein can promote simple, efficient, non-toxic delivery of oligonucleotides (e.g., siRNA, antisense oligonucleotide (ASO), macro-RNA), and promote potent silencing of therapeutic targets in kidney cells in vivo. In certain embodiments, cells in the kidney include, but are not limited to, tubule epithelial cells, macula densa cells, glomerular endothelial cells, podocytes, mesangial cells and parietal epithelial cells.

Unless otherwise specified, nomenclature used in connection with cell and tissue culture, molecular biology, immunology, microbiology, genetics, and protein and nucleic acid chemistry and hybridization described herein are those well-known and commonly used in the art. Unless otherwise specified, the methods and techniques provided herein are performed according to conventional methods well-known in the art and as described in various general and more specific references that are cited and discussed throughout the present specification. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

### Definitions

The use of the singular forms herein includes the plural unless specifically stated otherwise. As used herein, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and/or “consisting essentially of” are also provided.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein in the context of oligonucleotide sequences, “A” represents a nucleoside comprising the base adenine (e.g., adenosine or a chemically-modified derivative thereof), “G” represents a nucleoside comprising the base guanine (e.g., guanosine or a chemically-modified derivative thereof), “U” represents a nucleoside comprising the base uracil (e.g., uridine or a chemically-modified derivative thereof), and “C” represents a nucleoside comprising the base adenine (e.g., cytidine or a chemically-modified derivative thereof).

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. The siRNA is a duplex formed by a sense strand and antisense strand which have sufficient complementarity to each other to form said duplex. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” or “chemically modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide, which may be derivatized include: the 5 position, e.g., 5-(2-amino)propyl uridine, 5-bromo uridine, 5-propyne uridine, 5-propenyl uridine, etc.; the 6 position, e.g., 6-(2-amino)propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs, such as those described in Herdewijn, Antisense Nucleic Acid Drug Dev., 2000 Aug. 10(4):297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, or COOR, wherein R is substituted or unsubstituted C₁-C₆ alkyl, alkenyl, alkynyl, aryl, etc. Other possible modifications include those described in U.S. Pat. Nos. 5,858,988, and 6,291,438. In certain embodiments, the nucleotide analog comprises a 2′-O-methyl modification. In certain embodiments, the nucleotide analog comprises a 2′-fluoro modification.

The phosphate group of the nucleotide may also be modified, e.g., by substituting one or more of the oxygens of the phosphate group with sulfur (e.g., phosphorothioate), or by making other substitutions, which allow the nucleotide to perform its intended function, such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “RNA analog” refers to a polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Some RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An RNAi agent, e.g., an RNA silencing agent, having a strand, which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g., RNA interference (RNAi), transcriptional gene silencing (TGS), post-transcriptional gene silencing (PTGS), quelling, co-suppression, and translational repression) mediated by RNA molecules, which result in the inhibition or “silencing” of the expression of a corresponding protein-coding gene. RNA silencing has been observed in many types of organisms, including plants, animals, and fungi.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, a “target” refers to a particular nucleic acid sequence (e.g., a gene, an mRNA, a miRNA or the like) that an oligonucleotide or branched oligonucleotide of the disclosure binds to and/or otherwise effects the expression of. In certain embodiments, the target is expressed in the eye. In certain embodiments, target is expressed in a specific eye cell. In other embodiments, a target is associated with a particular disease or disorder in a subject.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g., mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g., an orthologue or paralogue) of the target gene. In some embodiments, the target gene is SLC5A2 (solute carrier family 5 member 2). The SLC5A2 gene is located on chromosome 16 and mainly expressed in the kidney, and encodes the sodium/glucose cotransporter 2 (SGLT2) protein The SGLT2 protein is responsible for glucose reabsorption in the kidney, 672 amino acids in length, and has a molecular mass of approximately 72, 897 Da.

As used herein, the term “RNA silencing agent” refers to an RNA, which is capable of inhibiting or “silencing” the expression of a target gene. In certain embodiments, the RNA silencing agent is capable of preventing complete processing (e.g., the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, antisense oligonucleotides, GAPMER molecules, and dual-function oligonucleotides, as well as precursors thereof. In one embodiment, the RNA silencing agent is capable of inducing RNA interference. In another embodiment, the RNA silencing agent is capable of mediating translational repression.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, 2N-methylguanosine and 2,2N,N-dimethylguanosine.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by a human. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

As used herein, the term “microRNA” (“miRNA”), also known in the art as “small temporal RNAs” (“stRNAs”), refers to a small (10-50 nucleotide) RNA, which are genetically encoded (e.g., by viral, mammalian, or plant genomes) and are capable of directing or mediating RNA silencing. An “miRNA disorder” shall refer to a disease or disorder characterized by an aberrant expression or activity of a miRNA.

As used herein, the term “dual functional oligonucleotide” refers to an RNA silencing agent having the formula T-L-μ, wherein T is an mRNA targeting moiety, L is a linking moiety, and μ is a miRNA recruiting moiety. As used herein, the terms “mRNA targeting moiety,” “targeting moiety,” “mRNA targeting portion” or “targeting portion” refer to a domain, portion or region of the dual functional oligonucleotide having sufficient size and sufficient complementarity to a portion or region of an mRNA chosen or targeted for silencing (i.e., the moiety has a sequence sufficient to capture the target mRNA).

As used herein, the term “linking moiety” or “linking portion” refers to a domain, portion or region of the RNA-silencing agent which covalently joins or links the mRNA.

As used herein, the term “antisense strand” of an RNA silencing agent, e.g., an siRNA, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target mRNA.

As used herein, the term “asymmetry,” as in the asymmetry of the duplex region of an RNA silencing agent (e.g., the stem of an shRNA), refers to an inequality of bond strength or base pairing strength between the termini of the RNA silencing agent (e.g., between terminal nucleotides on a first strand or stem portion and terminal nucleotides on an opposing second strand or stem portion), such that the 5′ end of one strand of the duplex is more frequently in a transient unpaired, e.g., single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, van der Waals interactions, and the like, between said nucleotides (or nucleotide analogs).

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein the term “destabilizing nucleotide” refers to a first nucleotide or nucleotide analog capable of forming a base pair with second nucleotide or nucleotide analog such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of an RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g., certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portion of universal nucleotides typically comprise a nitrogen-containing aromatic heterocyclic moiety.

As used herein, the terms “sufficient complementarity” or “sufficient degree of complementarity” mean that the RNA silencing agent has a sequence (e.g., in the antisense strand, mRNA targeting moiety or miRNA recruiting moiety), which is sufficient to bind the desired target RNA, respectively, and to trigger the RNA silencing of the target mRNA.

As used herein, the term “translational repression” refers to a selective inhibition of mRNA translation. Natural translational repression proceeds via miRNAs cleaved from shRNA precursors. Both RNAi and translational repression are mediated by RISC. Both RNAi and translational repression occur naturally or can be initiated by the hand of man, for example, to silence the expression of target genes.

Various methodologies of the instant disclosure include a step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the disclosure into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Various aspects of the disclosure are described in further detail in the following subsections.

Design of siRNA Molecules

Non-limiting examples of oligonucleotide include siRNA, antisense oligonucleotide (ASO), and macro-RNA.

In some embodiments, an siRNA molecule of the invention is a duplex consisting of a sense strand and complementary antisense strand, the antisense strand having sufficient complementary to a target sequence such as a mRNA sequence (e.g., a htt mRNA sequence, cyclophilin B mRNA sequence, etc.) to mediate RNAi. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is sufficiently complementary to a target region. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to a target sequence, and the other strand is identical or substantially identical to the first strand.

Generally, siRNAs can be designed by using any method known in the art, for instance, by using the following protocol:

1. The siRNA may be specific for a target sequence. Preferably, the first strand is substantially complementary to the target sequence, and the other strand is substantially complementary to the first strand. In an embodiment, the target sequence is outside a coding region of the target gene. Exemplary target sequences are selected from the 5′ untranslated region (5′-UTR) or an intronic region of a target gene. Cleavage of mRNA at these sites should eliminate translation of corresponding mutant protein. Target sequences from other regions of the htt gene are also suitable for targeting. A sense strand is designed based on the target sequence. Further, siRNAs with lower G/C content (35-55%) may be more active than those with G/C content higher than 55%. Thus, in one embodiment, the invention includes nucleic acid molecules having 35-55% G/C content.

2. The sense strand of the siRNA is designed based on the sequence of the selected target site. Preferably the RNA silencing agents of the invention do not elicit a PKR response (i.e., are of a sufficiently short length). However, longer RNA silencing agents may be useful, for example, in cell types incapable of generating a PRK response or in situations where the PKR response has been down-regulated or dampened by alternative means.

The siRNA molecules of the invention have sufficient complementarity with the target sequence such that the siRNA can mediate RNAi. In general, siRNA containing nucleotide sequences sufficiently identical to a target sequence portion of the target gene to effect RISC-mediated cleavage of the target gene are preferred. Accordingly, in a preferred embodiment, the sense strand of the siRNA is designed have to have a sequence sufficiently identical to a portion of the target. For example, the sense strand may have 100% identity to the target site. However, 100% identity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% identity, between the sense strand and the target RNA sequence is preferred. The invention has the advantage of being able to tolerate certain sequence variations to enhance efficiency and specificity of RNAi. In one embodiment, the sense strand has 4, 3, 2, 1, or 0 mismatched nucleotide(s) with a target region, such as a target region that differs by at least one base pair between a wild-type and mutant allele, e.g., a target region comprising the gain-of-function mutation, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., percent (%) homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The antisense or guide strand of the siRNA is routinely the same length as the sense strand and includes complementary nucleotides. In one embodiment, the guide and sense strands are fully complementary, i.e., the strands are blunt ended when aligned or annealed. In another embodiment, the strands of the siRNA can be paired in such a way as to have a 3′ overhang of 1 to 4, e.g., 2, nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus, in another embodiment, the nucleic acid molecules may have a 3′ overhang of 2 nucleotides, such as TT. The overhanging nucleotides may be either RNA or DNA. As noted above, it is desirable to choose a target region wherein the mutant:wild type mismatch is a purine:purine mismatch.

4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

5. Select one or more sequences that meet the criteria for evaluation.

Further general information about the design and use of siRNA may be found in “The siRNA User Guide,” available at The Max-Plank-Institut fur Biophysikalishe Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6 (log 10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

6. To validate the effectiveness by which siRNAs destroy target mRNAs (e.g., wild-type or mutant huntingtin mRNA), the siRNA may be incubated with target cDNA (e.g., huntingtin cDNA) in a Drosophila-based in vitro mRNA expression system. Radiolabeled with ³²P, newly synthesized target mRNAs (e.g., huntingtin mRNA) are detected autoradiographically on an agarose gel. The presence of cleaved target mRNA indicates mRNA nuclease activity. Suitable controls include omission of siRNA and use of non-target cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

siRNAs may be designed to target any of the target sequences described supra. Said siRNAs comprise an antisense strand which is sufficiently complementary with the target sequence to mediate silencing of the target sequence. In certain embodiments, the RNA silencing agent is a siRNA.

Sites of siRNA-mRNA complementation are selected which result in optimal mRNA specificity and maximal mRNA cleavage.

siRNA-Like Molecules

siRNA-like molecules of the invention have a sequence (i.e., have a strand having a sequence) that is “sufficiently complementary” to a target sequence of an mRNA (e.g., htt mRNA) to direct gene silencing either by RNAi or translational repression. siRNA-like molecules are designed in the same way as siRNA molecules, but the degree of sequence identity between the sense strand and target RNA approximates that observed between an miRNA and its target. In general, as the degree of sequence identity between a miRNA sequence and the corresponding target gene sequence is decreased, the tendency to mediate post-transcriptional gene silencing by translational repression rather than RNAi is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g., within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity. In one embodiment, where gene silencing by translational repression is desired, at least one non-identical nucleotide is present in the central portion of the complementarity site so that duplex formed by the miRNA guide strand and the target mRNA contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected such that it forms a wobble base pair (e.g., G:U) or a mismatched base pair (G:A, C:A, C:U, G:G, A:A, C:C, U:U). In a further preferred embodiment, the “bulge” is centered at nucleotide positions 12 and 13 from the 5′ end of the miRNA molecule.

### Modified RNA Silencing Agents

In certain aspects of the invention, an RNA silencing agent (or any portion thereof) of the invention as described supra may be modified such that the activity of the agent is further improved. For example, the RNA silencing agents described in above may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

### 1) Modifications to Enhance Target Discrimination

In certain embodiments, the RNA silencing agents of the invention may be substituted with a destabilizing nucleotide to enhance single nucleotide target discrimination (see U.S. application Ser. No. 11/698,689, filed Jan. 25, 2007, and U.S. Provisional Application No. 60/762,225 filed Jan. 25, 2006, both of which are incorporated herein by reference). Such a modification may be sufficient to abolish the specificity of the RNA silencing agent for a non-target mRNA (e.g., wild-type mRNA), without appreciably affecting the specificity of the RNA silencing agent for a target mRNA (e.g., gain-of-function mutant mRNA).

In preferred embodiments, the RNA silencing agents of the invention are modified by the introduction of at least one universal nucleotide in the antisense strand thereof. Universal nucleotides comprise base portions that are capable of base pairing indiscriminately with any of the four conventional nucleotide bases (e.g., A, G, C, U). A universal nucleotide is preferred because it has relatively minor effect on the stability of the RNA duplex, or the duplex formed by the guide strand of the RNA silencing agent and the target mRNA. Exemplary universal nucleotide includes those having an inosine base portion or an inosine analog base portion selected from the group consisting of deoxyinosine (e.g., 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In particularly preferred embodiments, the universal nucleotide is an inosine residue or a naturally occurring analog thereof.

In certain embodiments, the RNA silencing agents of the invention are modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destabilizing nucleotide and the specificity-determining nucleotide). In RNA silencing agents having two strands or strand portions (e.g., siRNAs and shRNAs), the destabilizing nucleotide may be introduced in the strand or strand portion that does not contain the specificity-determining nucleotide. In preferred embodiments, the destabilizing nucleotide is introduced in the same strand or strand portion that contains the specificity-determining nucleotide.

### 2) Modifications to Enhance Efficacy and Specificity

In certain embodiments, the RNA silencing agents of the invention may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the invention or an siRNA produced from a shRNA) into RISC in favor of the sense strand, such that the antisense strand preferentially guides cleavage or translational repression of a target mRNA, and thus increasing or improving the efficiency of target cleavage and silencing. Preferably the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the invention may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the invention may be enhanced such that there is at least one mismatched base pair between the 5′ end of the first or antisense strand and the 3′ end of the sense strand portion. Preferably, the mismatched base pair is selected from the group consisting of G:A, C:A, C:U, G:G, A:A, C:C and U:U. In another embodiment, the asymmetry of an RNA silencing agent of the invention may be enhanced such that there is at least one wobble base pair, e.g., G:U, between the 5′ end of the first or antisense strand and the 3′ end of the sense strand portion. In another embodiment, the asymmetry of an RNA silencing agent of the invention may be enhanced such that there is at least one base pair comprising a rare nucleotide, e.g., inosine (I). Preferably, the base pair is selected from the group consisting of an I:A, I:U and I:C. In yet another embodiment, the asymmetry of an RNA silencing agent of the invention may be enhanced such that there is at least one base pair comprising a modified nucleotide. In preferred embodiments, the modified nucleotide is selected from the group consisting of 2-amino-G, 2-amino-A, 2,6-diamino-G, and 2,6-diamino-A.

3) RNA Silencing Agents with Enhanced Stability

The RNA silencing agents of the present invention can be modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a preferred aspect, the invention features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In a preferred embodiment of the present invention, the RNA silencing agents may contain at least one modified nucleotide analogue. The nucleotide analogues may be located at positions where the target-specific silencing activity, e.g., the RNAi mediating activity or translational repression activity is not substantially effected, e.g., in a region at the 5′-end and/or the 3′-end of the siRNA molecule. Particularly, the ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In particular embodiments, the modifications are 2′-fluoro, 2′-amino and/or 2′-thio modifications. Particularly preferred modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moities of the instant invention. Additional modified residues include, deoxy-abasic, inosine, N3-methyl-uridine, N6,N6-dimethyl-adenosine, pseudouridine, purine ribonucleoside and ribavirin. In a particularly preferred embodiment, the 2′ moiety is a methyl group such that the linking moiety is a 2′-O-methyl oligonucleotide.

In an exemplary embodiment, the RNA silencing agent of the invention comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-O,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the invention comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone which is highly resistant to nuclease digestion and imparts improved binding specificity to the molecule (Nielsen, et al., Science, (2001), 254: 1497-1500).

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the invention includes RNA silencing agents having two complementary strands of nucleic acid, wherein the two strands are crosslinked. The invention also includes RNA silencing agents which are conjugated or unconjugated (e.g., at its 3′ terminus) to another moiety (e.g., a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell or improve the stability of the siRNA derivative compared to the corresponding siRNA.

Other exemplary modifications include: (a) 2′ modification, e.g., provision of a 2′ OMe moiety on a U in a sense or antisense strand, but especially on a sense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an 0 with an S, in the phosphate backbone, e.g., the provision of a phosphorothioate modification, on the U or the A or both, especially on an antisense strand; e.g., with the replacement of a P with an S; (c) replacement of the U with a C5 amino linker; (d) replacement of an A with a G (sequence changes are preferred to be located on the sense strand and not the antisense strand); and (d) modification at the 2′, 6′, 7?, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ 0 Me moiety and modification of the backbone, e.g., with the replacement of a P with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### Oligonucleotide Conjugate

The oligonucleotide described herein can be an oligonucleotide conjugate linked to a functional moiety. A functional moiety is a molecule that confers one or more additional activities to the RNA silencing agent. In certain embodiments, the functional moieties enhance cellular uptake by target cells (e.g., neuronal cells). Thus, the disclosure includes RNA silencing agents which are conjugated or unconjugated (e.g., at its 5′ and/or 3′ terminus) to another moiety (e.g., a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a certain embodiment, the functional moiety is a hydrophobic moiety. In a certain embodiment, the hydrophobic moiety is selected from the group consisting of fatty acids, steroids, secosteroids, lipids, gangliosides and nucleoside analogs, endocannabinoids, and vitamins. In a certain embodiment, the steroid selected from the group consisting of cholesterol and Lithocholic acid (LCA). In a certain embodiment, the fatty acid selected from the group consisting of Eicosapentaenoic acid (EPA), Docosahexaenoic acid (DHA) and Docosanoic acid (DCA). In a certain embodiment, the vitamin selected from the group consisting of choline, vitamin A, vitamin E, and derivatives or metabolites thereof. In a certain embodiment, the vitamin is selected from the group consisting of retinoic acid and alpha-tocopheryl succinate.

In a certain embodiment, an RNA silencing agent of disclosure is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the Y end of the sense strand. In certain embodiments, the lipophilic moiety is selected from the group consisting of cholesterol, vitamin E, vitamin K, vitamin A, folic acid, a cationic dye (e.g., Cy3). In an exemplary embodiment, the lipophilic moiety is cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine.

In certain embodiments, the functional moieties may comprise one or more ligands tethered to an RNA silencing agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These can be located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., 0-phenanthroline), a polyamine, a tripeptide (e.g., lys-tyr-lys tripeptide), or a metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to an RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine, has an increased affinity for the HIV Rev-response element (RRE). In some embodiments, the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, either directly or indirectly, via an intervening tether, to a ligand-conjugated carrier. In certain embodiments, the coupling is through a covalent bond. In certain embodiments, the ligand is attached to the carrier via an intervening tether. In certain embodiments, a ligand alters the distribution, targeting or lifetime of an RNA silencing agent into which it is incorporated. In certain embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine (GalNAc) or derivatives thereof, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g., acridines and substituted acridines), cross-linkers (e.g., psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycosides, artificial endonucleases (e.g., EDTA), lipophilic molecules, e.g., cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C10, C11, C12, C13, C14, C15, C16, C17, C18, C19, or C20 fatty acids) and ethers thereof, e.g., C10, C11, C12, C13, C14, C15, C16, C17, C18, C19, or C20 alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP or AP. In certain embodiments, the ligand is GalNAc or a derivative thereof.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-kB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNFα), interleukin-1 beta, or gamma interferon. In one aspect, the ligand is a lipid or lipid-based molecule. Such a lipid or lipid-based molecule can bind a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid-based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a certain embodiment, the lipid-based ligand binds HSA. A lipid-based ligand can bind HSA with a sufficient affinity such that the conjugate will be distributed to a non-kidney tissue. However, it is contemplated that the affinity not be so strong that the HSA-ligand binding cannot be reversed. In another embodiment, the lipid-based ligand binds HSA weakly or not at all, such that the conjugate will be distributed to the kidney. Other moieties that target to kidney cells can also be used in place of or in addition to the lipid-based ligand.

In another aspect, the ligand is a moiety, e.g., a vitamin, which is taken up by a target cell, e.g., a proliferating cell. These can be useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low-density lipoprotein (LDL).

In another aspect, the ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent can be an alpha-helical agent, which may have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

Structures of exemplary functional moieties are depicted below.

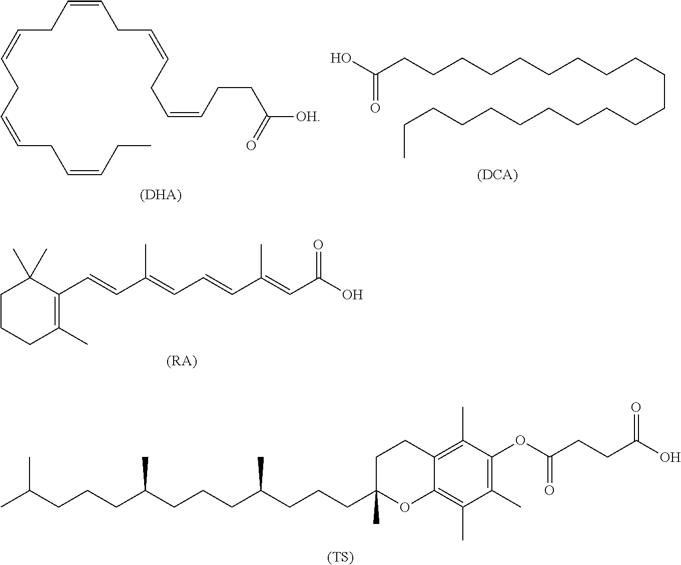

In certain embodiments, two DHA functional moieties are linked to the oligonucleotide.

In certain embodiments, the oligonucleotide comprises an antisense oligonucleotide or an siRNA.

In certain embodiments, the siRNA comprises a sense strand and an antisense strand. In certain embodiments, the antisense strand comprises about 15 nucleotides to 25 nucleotides in length (e.g., 15, 16, 17, 18, 19, 20, 21, 22, 23, 24, or 25 nucleotides in length). In certain embodiments, the antisense strand is 20 nucleotides in length, 21 nucleotides in length, or 22 nucleotides in length. In certain embodiments, the sense strand comprises about 15 nucleotides to 25 nucleotides in length (e.g., 15, 16, 17, 18, 19, 20, 21, 22, 23, 24, or 25 nucleotides in length). In certain embodiments, the sense strand is 15 nucleotides in length, 16 nucleotides in length, 18 nucleotides in length, or 20 nucleotides in length.

In certain embodiments, the siRNA comprises a double-stranded region of 15 base pairs to 20 base pairs (e.g., 15, 16, 17, 18, 19, or 20 base pairs). In certain embodiments, the siRNA comprises a double-stranded region of 15 base pairs, 16 base pairs, 18 base pairs, or 20 base pairs.

In certain embodiments, the siRNA comprises at least one blunt-end. In certain embodiments, the siRNA comprises two blunt-ends.

In certain embodiments, the siRNA comprises at least one single stranded nucleotide overhang (also referred to herein as a “single-stranded tail”). In certain embodiments, the siRNA comprises two single stranded nucleotide overhangs. In certain embodiments, the siRNA comprises about a 2-nucleotide to 5-nucleotide single stranded nucleotide overhang (e.g., a 2-, 3-, 4-, or 5-nucleotide overhang). In certain embodiments, the siRNA comprises a 2-nucleotide single stranded nucleotide overhang or a 5-nucleotide single stranded nucleotide overhang.

In certain embodiments, the siRNA comprises naturally occurring nucleotides (i.e., unmodified ribonucleotides).

In certain embodiments, the siRNA comprises at least one modified nucleotide. In certain embodiments, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the siRNA comprises at least one modified internucleotide linkage. In certain embodiments, the modified internucleotide linkage comprises a phosphorothioate internucleotide linkage. In certain embodiments, the siRNA comprises 4-16 phosphorothioate internucleotide linkages. In certain embodiments, the siRNA comprises 8-13 phosphorothioate internucleotide linkages.

In certain embodiments, the siRNA comprises at least 80% chemically modified nucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% chemically modified nucleotides). In certain embodiments, the siRNA is fully chemically modified.

In certain embodiments, the siRNA comprises at least 70% 2′-O-methyl nucleotide modifications (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the antisense strand comprises at least 70% 2′-O-methyl nucleotide modifications (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the antisense strand comprises about 70% to 90% 2′-O-methyl nucleotide modifications. In certain embodiments, the sense strand comprises at least 65% 2′-O-methyl nucleotide modifications (e.g., 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the sense strand comprises 100% 2′-O-methyl nucleotide modifications.

In certain embodiments, the sense strand comprises one or more nucleotide mismatches between the antisense strand and the sense strand.

In certain embodiments, the antisense strand comprises a 5′ phosphate, a 5′-alkyl phosphonate, a 5′ alkylene phosphonate, or a 5′ alkenyl phosphonate. In certain embodiments, the antisense strand comprises a 5′ vinyl phosphonate.

In certain embodiments, the functional moiety is linked to the 5′ end and/or 3′ end of the oligonucleotide. In certain embodiments, the functional moiety is linked to the 5′ end and/or 3′ end of the sense strand or to the 5′ end and/or 3′ end of the antisense strand. In certain embodiments, the functional moiety is linked to the 3′ end of the sense strand.

In certain embodiments, the functional moiety is linked to the RNA silencing agent by a linker. In certain embodiments, the functional moiety is linked to the antisense strand and/or sense strand by a linker. In certain embodiments, the functional moiety is linked to the 3′ end of a sense strand by a linker. In certain embodiments, the linker is a cleavable linker. In certain embodiments, the cleavable linker comprises a phosphodiester linkage, a disulfide linkage, an acid-labile linkage, or a photocleavable linkage.

In certain embodiments, the cleavable linker comprises a dTdT dinucleotide with phosphodiester internucleotide linkages.

In certain embodiments, the acid-labile linkage comprises a β-thiopropionate linkage or a carboxydimethylmaleic anhydride (CDM) linkage.

In certain embodiments, the linker comprises a divalent or trivalent linker.

In certain embodiments, the divalent or trivalent linker is selected from the group consisting of:

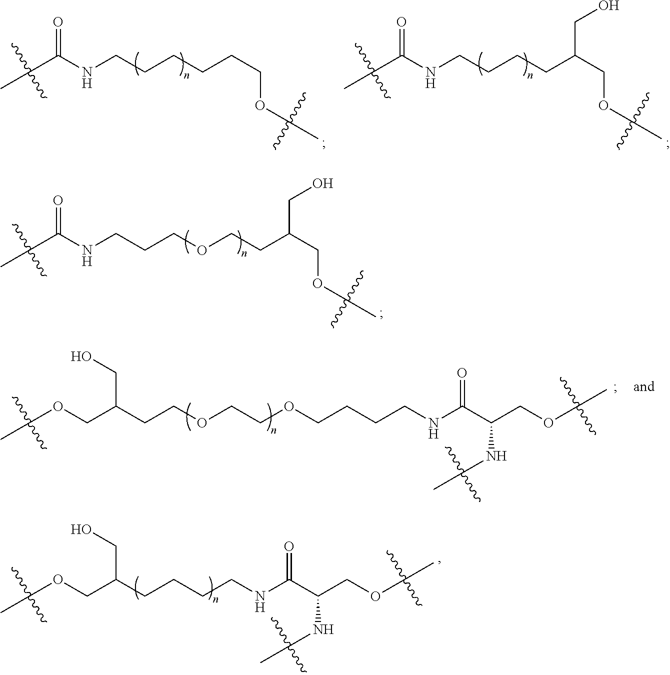

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, when the linker is a trivalent linker, the linker further links a phosphodiester or phosphodiester derivative.

In certain embodiments, the phosphodiester or phosphodiester derivative is selected from the group consisting of:

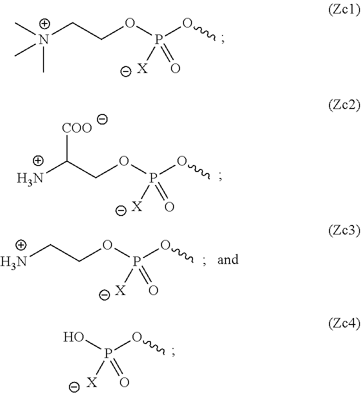

wherein X is O, S or BH₃.

The above recited moiety Zc1 is phosphatidylcholine (PC). Any one of the functional moieties described herein may comprise a phosphatidylcholine (PC) esterified derivative, i.e., phosphatidylcholine (PC) esterified retinoic acid (PC-RA), phosphatidylcholine (PC) esterified docosahexaenoic acid (PC-DHA), phosphatidylcholine (PC) esterified docosanoic acid (PC-DCA), or phosphatidylcholine (PC) esterified α-tocopheryl succinate (PC-TS).

In certain embodiments, the nucleotides at positions 1 and 2 from the 3′ end of sense strand, and the nucleotides at positions 1 and 2 from the 5′ end of antisense strand are connected to adjacent ribonucleotides via phosphorothioate linkages.

In certain embodiments, the oligonucleotide conjugate comprises the structure:

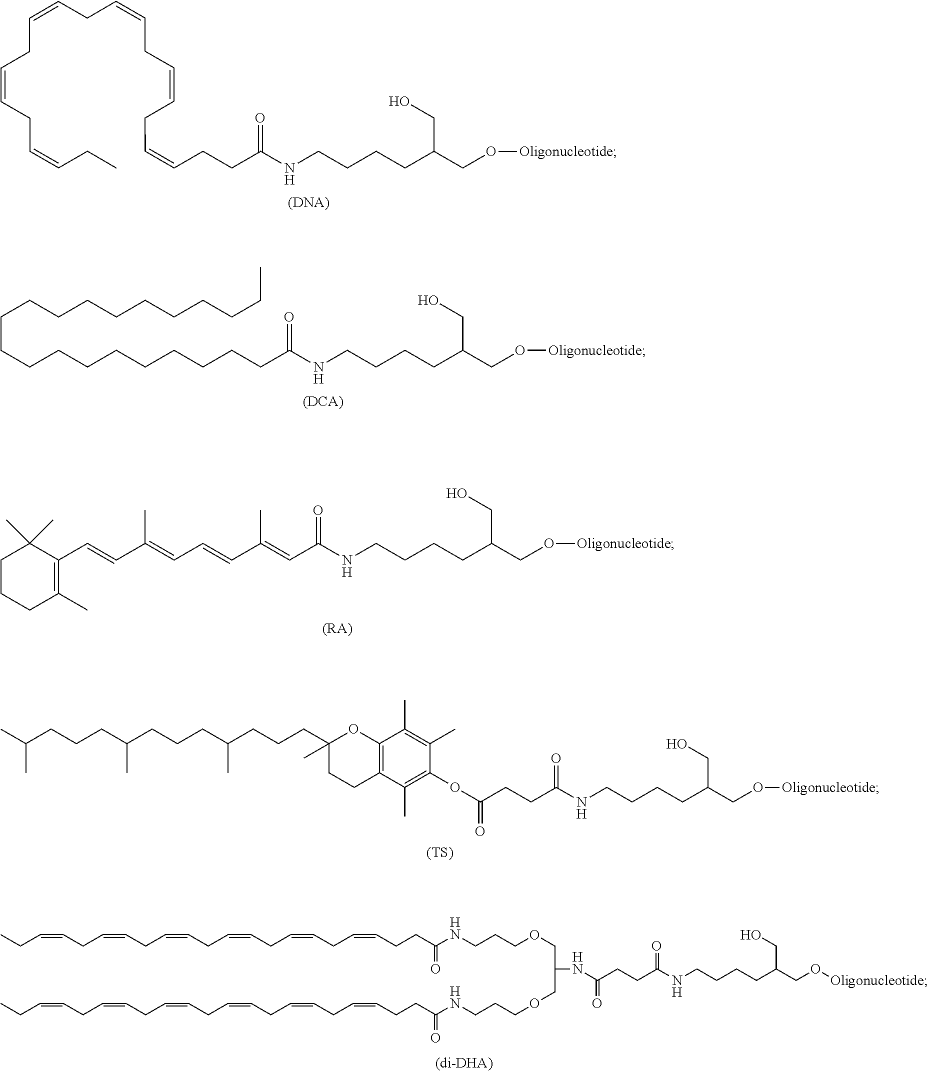

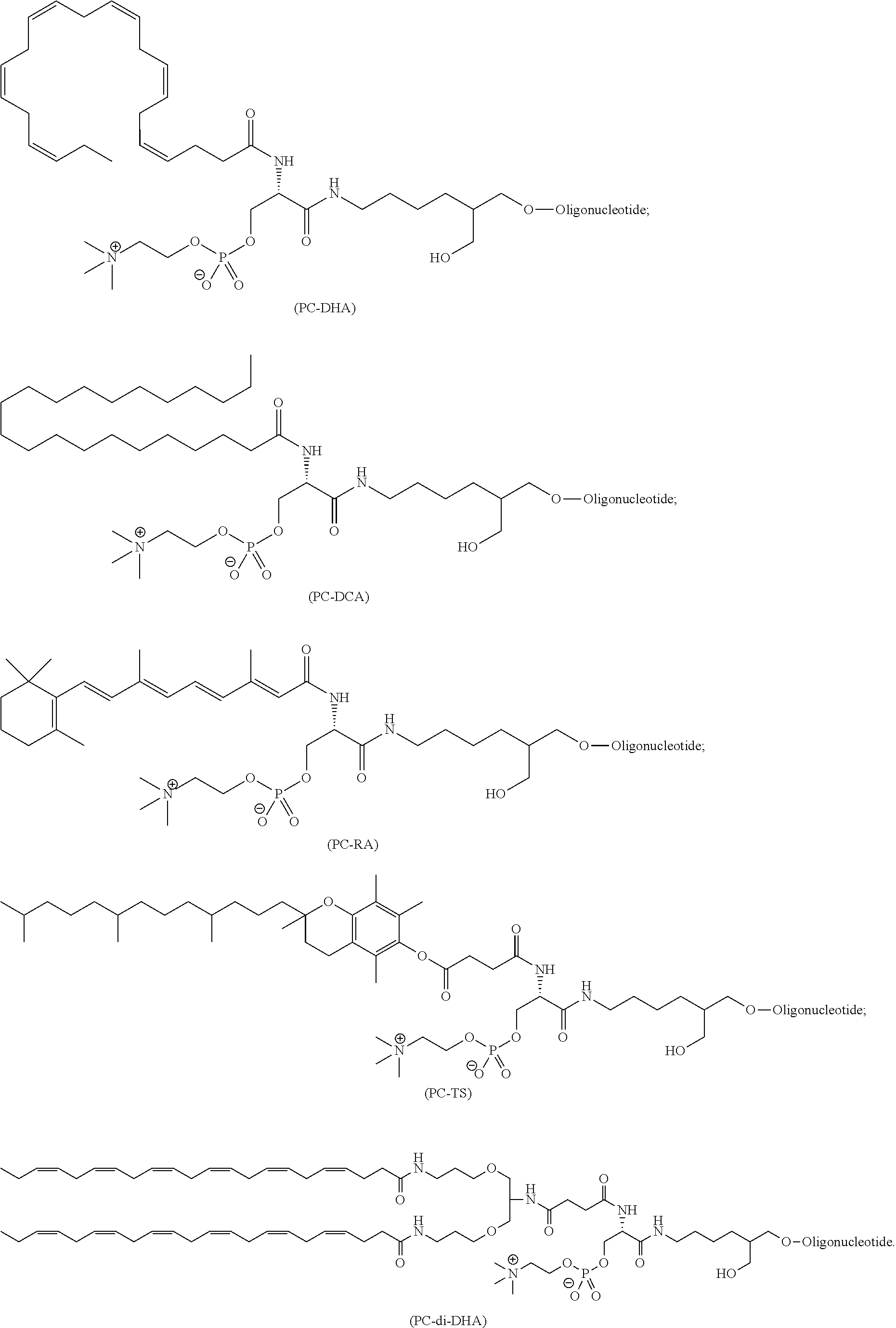

For any of the above recited structures, the term “oligonucleotide” corresponds to any of the oligonucleotides recited herein, e.g., an ASO or siRNA. In certain embodiments, the term “oligonucleotide” in the structures recited above corresponds to the sense strand of an siRNA. In certain embodiments, the oxygen immediately adjacent to the term “oligonucleotide” in the structures is linked to the 3′ end of a sense strand of an siRNA.

The various functional moieties of the disclosure and means to conjugate them to RNA silencing agents are described in further detail in WO2017/030973A1 and WO2018/031933A2, incorporated herein by reference.

### Branched Oligonucleotides

The branched oligonucleotides described here comprise two or more oligonucleotides linked together. The different branched oligonucleotides described herein (e.g., a branched oligonucleotide with two, three, or four oligonucleotides) enhanced topical delivery of the oligonucleotide.

In certain embodiments, one or more of the oligonucleotides of the branched oligonucleotide further comprises a functional moiety linked to the oligonucleotide, wherein the functional moiety comprises any one of retinoic acid, DHA, DCA, or α-tocopheryl succinate. The functional moieties as described above in the Oligonucleotide Conjugate section can be applied to the oligonucleotides of the branched oligonucleotides. Similarly, the oligonucleotides as described above in the Oligonucleotide Conjugate section can serve as the oligonucleotides of the branched oligonucleotides, including type (ASO or siRNA), strand length, and chemical modifications.

In certain embodiments, the two or more oligonucleotides in the branched oligonucleotide are connected to one another by one or more moieties independently selected from a linker, a spacer and a branching point.

In certain embodiments, the linker comprises an ethylene glycol chain, an alkyl chain, a peptide, an RNA, a DNA, a phosphate, a phosphonate, a phosphoramidate, an ester, an amide, a triazole, or combinations thereof.

In certain embodiments, the branching point comprises a polyvalent organic species or derivative thereof.

In another embodiment, the branching point is an amino acid derivative. In another embodiment of the branching point is selected from the formulas of:

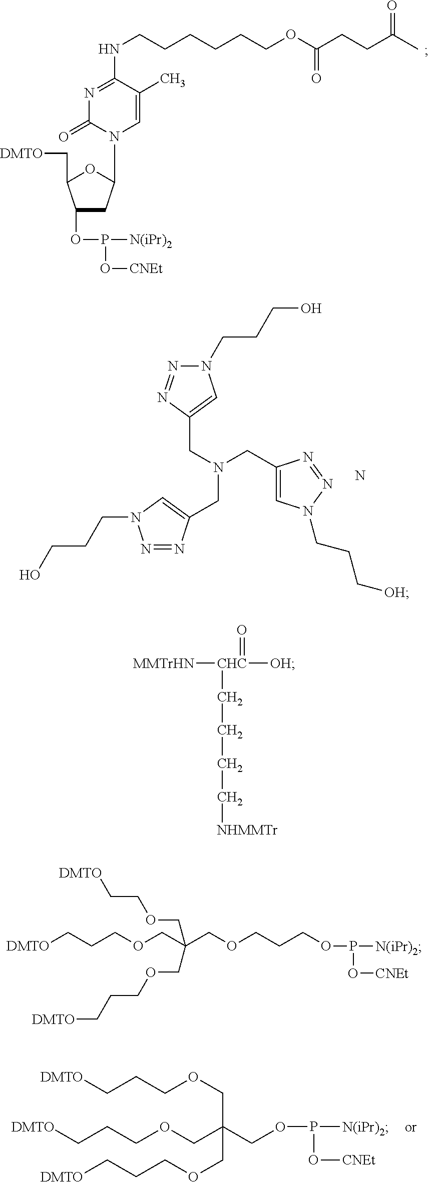

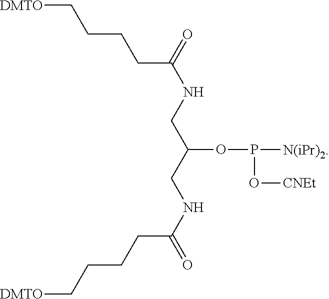

Polyvalent organic species are moieties comprising carbon and three or more valencies (i.e., points of attachment with moieties such as S, L or N, as defined above). Non-limiting examples of polyvalent organic species include triols (e.g., glycerol, phloroglucinol, and the like), tetrols (e.g., ribose, pentaerythritol, 1,2,3,5-tetrahydroxybenzene, and the like), tri-carboxylic acids (e.g., citric acid, 1,3,5-cyclohexanetricarboxylic acid, trimesic acid, and the like), tetra-carboxylic acids (e.g., ethylenediaminetetraacetic acid, pyromellitic acid, and the like), tertiary amines (e.g., tripropargylamine, triethanolamine, and the like), triamines (e.g., diethylenetriamine and the like), tetramines, and species comprising a combination of hydroxyl, thiol, amino, and/or carboxyl moieties (e.g., amino acids such as lysine, serine, cysteine, and the like).

In certain embodiments, the spacer comprises an ethylene glycol chain, an alkyl chain, a peptide, an RNA, a DNA, a phosphate, a phosphonate, a phosphoramidate, an ester, an amide, a triazole, or a combination thereof.

In certain embodiments, the linker comprises the structure L1:

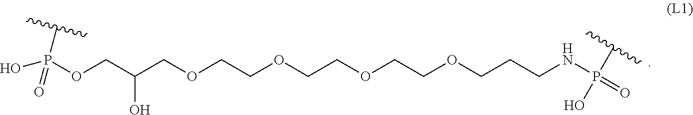 [SMILES: *P(=O)(O)NCCCOCCOCCOCCOCC(O)COP(*)(=O)O]

In certain embodiments, the linker comprises the structure L2:

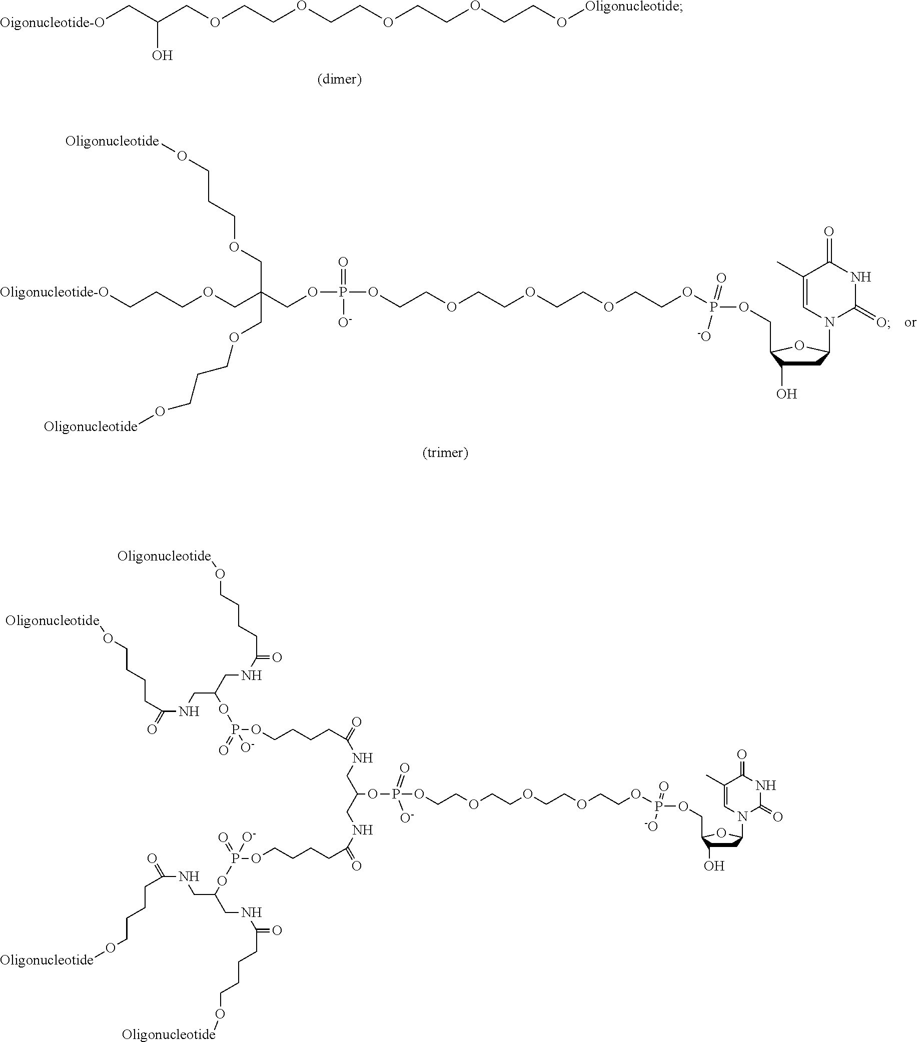

In certain embodiments, the branched oligonucleotide consists of two oligonucleotides. In certain embodiments, the branched oligonucleotide consists of three oligonucleotides. In certain embodiments, the branched oligonucleotide consists of four oligonucleotides. In certain embodiments, the oligonucleotides are siRNA.

In certain embodiments, the branched oligonucleotide comprises the structure:

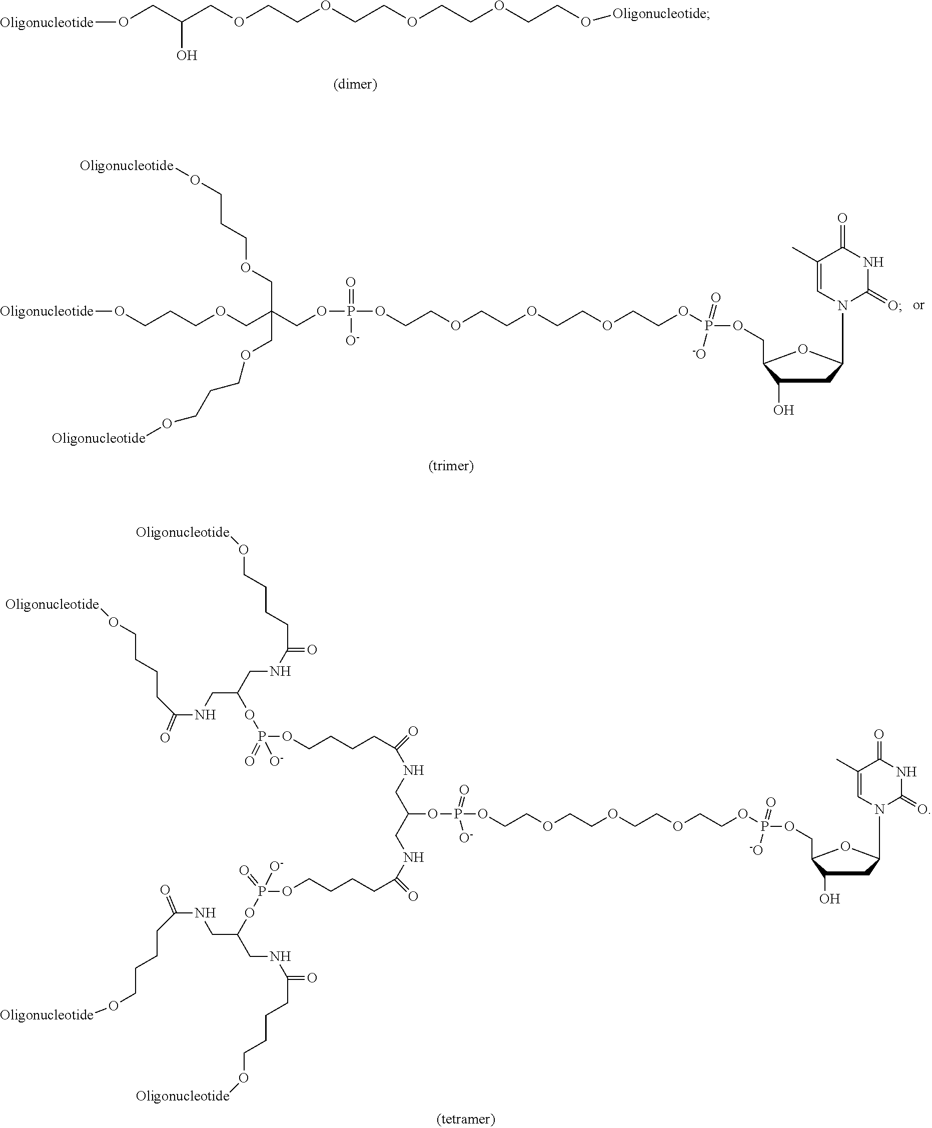

For any of the above recited structures, the term “oligonucleotide” corresponds to any of the oligonucleotides recited herein, e.g., an ASO or siRNA. In certain embodiments, the term “oligonucleotide” in the structures recited above corresponds to the sense strand of an siRNA. In certain embodiments, the oxygen immediately adjacent to the term “oligonucleotide” in the structures is linked to the 3′ end of a sense strand of an siRNA.

Branched oligonucleotides, including synthesis and methods of use, are described in greater detail in WO2017/132669, incorporated herein by reference.

### Branched Oligonucleotide

In certain embodiments, the oligonucleotide is a branched oligonucleotide.

In certain embodiments, the linker comprises an ethylene glycol chain, an alkyl chain, a peptide, an RNA, a DNA, a phosphate, a phosphonate, a phosphoramidate, an ester, an amide, a triazole, or any combinations thereof.

In certain embodiments, the branching point comprises a polyvalent organic species or derivative thereof.

In another embodiment, the branching point is an amino acid derivative. In another embodiment of the branching point is selected from the formulas of:

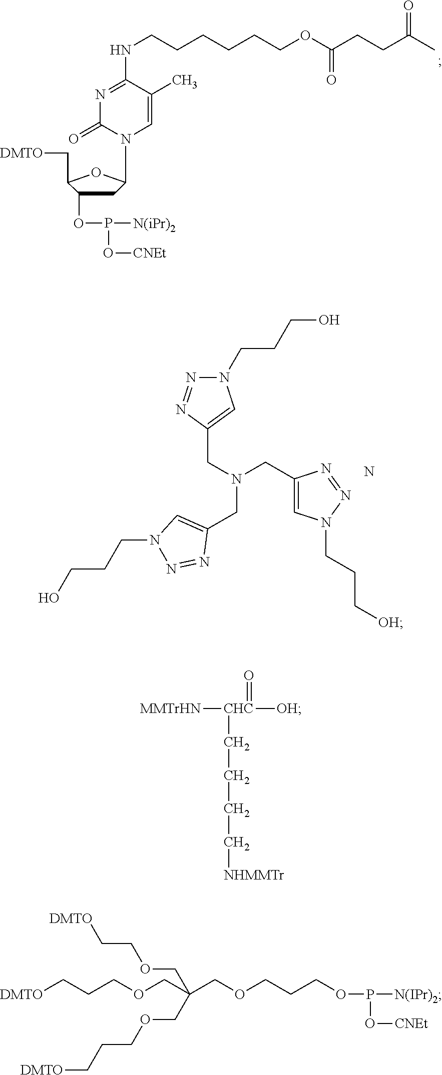

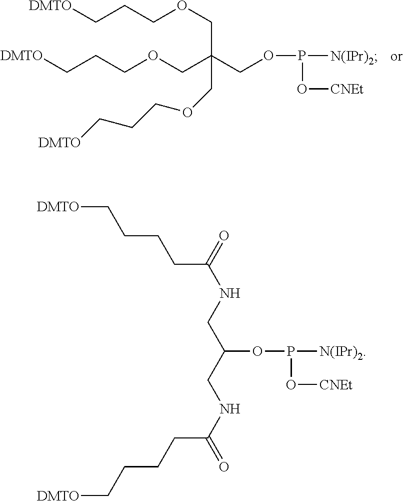

Polyvalent organic species are moieties comprising carbon and three or more valencies (i.e., points of attachment with moieties such as S, L or N, as defined above). Non-limiting examples of polyvalent organic species include triols (e.g., glycerol, phloroglucinol, and the like), tetrols (e.g., ribose, pentaerythritol, 1,2,3,5-tetrahydroxybenzene, and the like), tri-carboxylic acids (e.g., citric acid, 1,3,5-cyclohexanetricarboxylic acid, trimesic acid, and the like), tetra-carboxylic acids (e.g., ethylenediaminetetraacetic acid, pyromellitic acid, and the like), tertiary amines (e.g., tripropargylamine, triethanolamine, and the like), triamines (e.g., diethylenetriamine and the like), tetramines, and species comprising a combination of hydroxyl, thiol, amino, and/or carboxyl moieties (e.g., amino acids such as lysine, serine, cysteine, and the like).

In certain embodiments, the spacer comprises an ethylene glycol chain, an alkyl chain, a peptide, an RNA, a DNA, a phosphate, a phosphonate, a phosphoramidate, an ester, an amide, a triazole, or a combination thereof.

### Internucleotide Linkage Modifications

In certain embodiments, at least one internucleotide linkage, intersubunit linkage, or nucleotide backbone is modified in the RNA silencing agent. In certain embodiments, all of the internucleotide linkages in the RNA silencing agent are modified. In certain embodiments, the modified internucleotide linkage comprises a phosphorothioate internucleotide linkage. In certain embodiments, the RNA silencing agent comprise 1, 2, 3, 4, 5, 6, 7, 8, 9, 10, 11, 12, 13, 14, 15, 16, 17, 18, 19, 20, 21, 22, 23, 24, or 25 phosphorothioate internucleotide linkages. In certain embodiments, the RNA silencing agent comprises 4-16 phosphorothioate internucleotide linkages. In certain embodiments, the RNA silencing agent comprises 8-13 phosphorothioate internucleotide linkages. In certain embodiments, the RNA silencing agent is a dsRNA comprising an antisense strand and a sense strand, each comprising a 5′ end and a 3′ end. In certain embodiments, the nucleotides at positions 1 and 2 from the 5′ end of sense strand are connected to adjacent ribonucleotides via phosphorothioate internucleotide linkages. In certain embodiments, the nucleotides at positions 1 and 2 from the 3′ end of sense strand are connected to adjacent ribonucleotides via phosphorothioate internucleotide linkages. In certain embodiments, the nucleotides at positions 1 and 2 from the 5′ end of antisense strand are connected to adjacent ribonucleotides via phosphorothioate internucleotide linkages. In certain embodiments, the nucleotides at positions 1-2 to 1-8 from the 3′ end of antisense strand are connected to adjacent ribonucleotides via phosphorothioate internucleotide linkages. In certain embodiments, the nucleotides at positions 1-2, 1-3, 1-4, 1-5, 1-6, 1-7, or 1-8 from the 3′ end of antisense strand are connected to adjacent ribonucleotides via phosphorothioate internucleotide linkages. In certain embodiments, the nucleotides at positions 1-2 to 1-7 from the 3′ end of antisense strand are connected to adjacent ribonucleotides via phosphorothioate internucleotide linkages.

In certain embodiments, the oligonucleotide described herein contains at least one of the backbone modification Extended Nucleic Acid (exNA). This chemical modification of the backbone significantly enhances oligonucleotide metabolic stability. The chemical modification includes one or more carbon atoms or chains inserted in the backbone at the 5′-position, 3′-position, or both. This structural modulation forms non-canonical stretched/flexible structure on oligo-backbones, which protect oligonucleotides from cleavage by various nucleases.

The synthesis protocol and structure for exNA-modified oligonucleotides is described in WO2021242883, WO2021195533, and WO2020198509, each of which is incorporated herein by reference. Importantly, the exNA monomer phosphoramidite synthesis can be realized from commercially available nucleosides and the exNA-modified oligonucleotide can be made using conventional oligonucleotide solid phase synthesis procedures on an automatic oligo synthesizer.

This synthetic procedure provides following noteworthy benefits. For example, the conversion of a regular nucleoside to an “exNA-format” is applicable to many diverse modified nucleosides. Thus, this expands the possibilities to synthesize and create many more types of modified oligonucleotides with compatibility of the chemical synthesis. Secondly, there is no need of a separate specific synthesis procedure during an oligonucleotide synthesis cycle. This is a huge benefit in the ease of use of these oligos, especially with an automated synthesizer where a bottle of exNA phosphoramidite could easily be added to the machine. Thirdly, there is no need of a specific oligonucleotide deprotection condition because the exNA phosphoramidites and oligos are compatible with conventional deprotection conditions. Again, this is beneficial for the ease of synthesis and in the use of an automated synthesizer. Fourthly, it is possible to synthesize mix-mer oligonucleotide having both exNA and clinically validated modified nucleotides (e.g., 2′-OMe, 2′-F, phosphorothioate, various ligand conjugates, lipid conjugates, etc.).

In one aspect, the disclosure provides a modified oligonucleotide, said oligonucleotide having a 5′ end, a 3′ end, that is complementary to a target, wherein the oligonucleotide comprises at least one modified intersubunit linkage of Formula Ia:

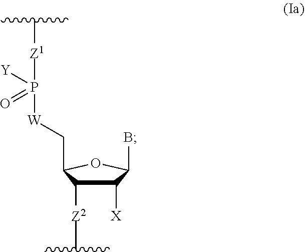

wherein:
B is a base moiety;
W is O or O(CH₂)ₙ¹, wherein n¹ is 1 to 10;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z¹ is O or O(CH₂)ₙ², wherein n² is 1 to 10;
Z² is O or O(CH₂)ₙ³, wherein n³ is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula Ia, Z₁ is O(CH₂)ₙ², wherein n² is 1 to 10 and W is O. In an embodiment of Formula Ia, Z₁ is O and W is O(CH₂)ₙ¹, wherein n¹ is 1 to 10. In an embodiment of Formula Ia, Z₁ is O(CH₂)ₙ², wherein n² is 1 to 10 and W is O(CH₂)ₙ¹, wherein n¹ is 1 to 10.

In an embodiment of Formula Ia, Z¹ is O(CH₂)ₙ², n² is 1, W is O, and Y is O⁻. In an embodiment of Formula Ia, Z¹ is O, W is O(CH₂)ₙ¹, n¹ is 1, and Y is O. In an embodiment of Formula Ia, Z¹ is O(CH₂)ₙ², n² is 1, W is O, and Y is O⁻. In an embodiment of Formula Ia, Z¹ is O(CH₂)ₙ², n² is 1, W is O(CH₂)ₙ¹, and Y is O⁻. In an embodiment of Formula Ia, Z¹ is O(CH₂)ₙ², n² is 1, W is O(CH₂)ₙ¹, and Y is S⁻.

In an embodiment of Formula Ia, Y is S⁻. In an embodiment of Formula Ia, X is OR¹ or F.

In an embodiment of Formula Ia, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula (I):

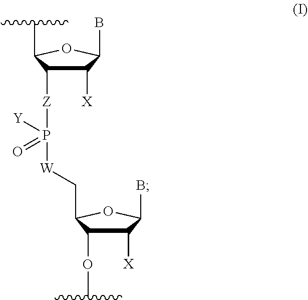

wherein:
B is a base moiety;
W is O or O(CH₂)ₙ¹, wherein n¹ is 1 to 10;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ², wherein n² is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula I, Z is O(CH₂)ₙ², wherein n² is 1 to 10 and W is O. In an embodiment of Formula I, Z is O and W is O(CH₂)ₙ¹, wherein n¹ is 1 to 10. In an embodiment of Formula I, Z is O(CH₂)ₙ², wherein n² is 1 to 10 and W is O(CH₂)ₙ¹, wherein n¹ is 1 to 10.

In an embodiment of Formula I, Z is O(CH₂)ₙ², n² is 1, W is O, and Y is O⁻. In an embodiment of Formula I, Z is O, W is O(CH₂)ₙ¹, n¹ is 1, and Y is O⁻. In an embodiment of Formula I, Z is O(CH₂)ₙ², n² is 1, W is O, and Y is O⁻. In an embodiment of Formula I, Z is O(CH₂)ₙ², n² is 1, W is O(CH₂)ₙ¹, and Y is O⁻. In an embodiment of Formula I, Z is O(CH₂)ₙ², n² is 1, W is O(CH₂)ₙ¹, and Y is S⁻.

In an embodiment of Formula I, Y is S⁻. In an embodiment of Formula I, X is OR¹ or F.

In an embodiment of Formula I, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula IIa:

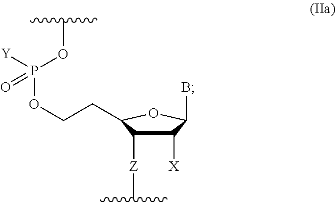

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ², wherein n² is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula IIa, Y is S⁻. In an embodiment of Formula IIa, Y is O. In an embodiment of Formula IIa, X is OR¹ or F.

In an embodiment of Formula IIa, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula II:

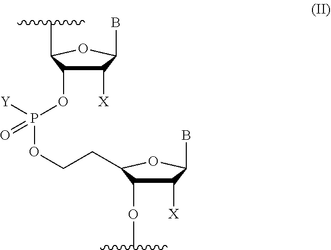

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ², wherein n² is 1 to 10;
R¹ is a substituted or unsubstituted C₁-C₆ alkyl, alkenyl, alkynyl, or aryl, or mixtures thereof;
R² is a substituted or unsubstituted C₁-C₆ alkyl, alkenyl, alkynyl, or aryl, or mixtures thereof.

In an embodiment of Formula II, Y is S⁻. In an embodiment of Formula II, Y is O. In an embodiment of Formula II, X is OR¹ or F.

In an embodiment of Formula II, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula IIIa:

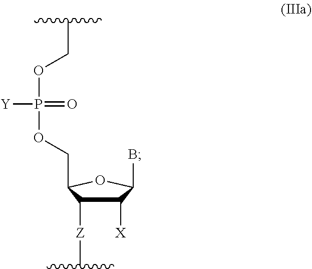

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ², wherein n² is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula IIIa, Y is S⁻. In an embodiment of Formula IIIa, X is OR¹ or F.

In an embodiment of Formula IIIa, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula III:

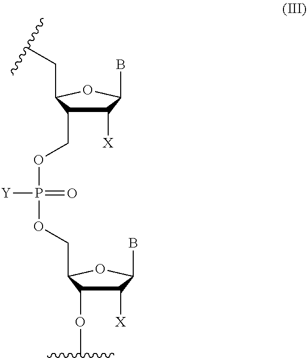

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ², wherein n² is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula III, Y is S⁻. In an embodiment of Formula III, X is OR¹ or F.

In an embodiment of Formula III, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula IVa:

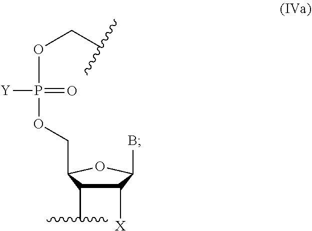

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH; Z is O or O(CH₂)ₙ², wherein n² is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula IVa, Y is S⁻. In an embodiment of Formula IVa, X is OR¹ or F.

In an embodiment of Formula IVa, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula IV:

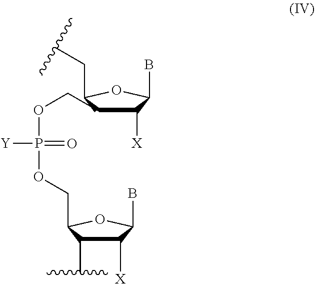

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula IV, Y is S⁻. In an embodiment of Formula IV, X is OR¹ or F.

In an embodiment of Formula IV, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula Va:

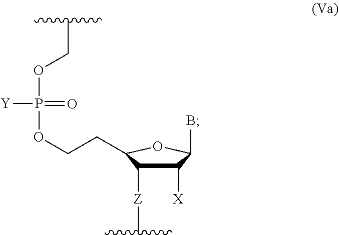

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ², wherein n² is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula Va, Y is S⁻. In an embodiment of Formula Va, X is OR¹ or F.

In an embodiment of Formula Va, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula V:

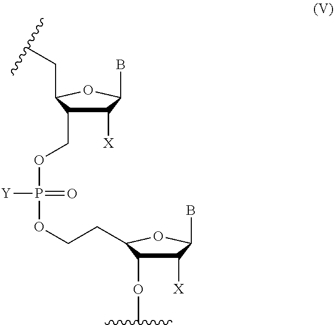

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ², wherein n² is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula V, Y is S⁻. In an embodiment of Formula V, X is OR¹ or F.

In an embodiment of Formula V, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula VIa:

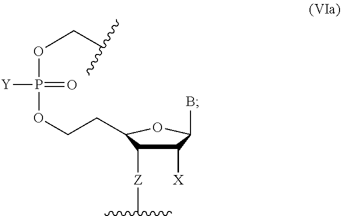

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ², wherein n² is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula VIa, Y is S⁻. In an embodiment of Formula VIa, X is OR¹ or F.

In an embodiment of Formula VIa, the base moiety B is selected from the group consisting of adenine, guanine, cytosine, and uracil.

In another aspect, the disclosure provides a modified oligonucleotide, said oligonucleotide having a 5′ end, a 3′ end, that is complementary to a target, wherein the oligonucleotide comprises at least one modified intersubunit linkage of Formula VI:

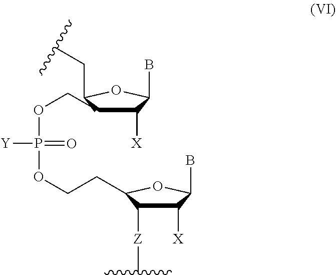

wherein:
B is a base moiety;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z is O or O(CH₂)ₙ², wherein n² is 1 to 10;
R¹ is a substituted or unsubstituted C₁-C₆ alkyl, alkenyl, alkynyl, or aryl, or mixtures thereof; and
R² is a substituted or unsubstituted C₁-C₆ alkyl, alkenyl, alkynyl, or aryl, or mixtures thereof.

In an embodiment of Formula VI, Y is S⁻. In an embodiment of Formula VI, X is OR¹ or F.

In an embodiment of Formula VI, the base moiety B is selected from the group consisting of adenine, guanine, cytosine, and uracil.

In certain embodiments, the oligonucleotide of the disclosure comprises at least one (e.g., 1, 2, 3, 4, 5, 6, 7, 8, 9, 10, or more) exNA internucleotide linkage of Formula Ia. In certain embodiments, the oligonucleotide of the disclosure comprises at least one (e.g., 1, 2, 3, 4, 5, 6, 7, 8, 9, 10, or more) exNA internucleotide linkage of Formula I. In certain embodiments, the oligonucleotide of the disclosure comprises at least one (e.g., 1, 2, 3, 4, 5, 6, 7, 8, 9, 10, or more) exNA internucleotide linkage of Formula IIa. In certain embodiments, the oligonucleotide of the disclosure comprises at least one (e.g., 1, 2, 3, 4, 5, 6, 7, 8, 9, 10, or more) exNA internucleotide linkage of Formula II. In certain embodiments, the oligonucleotide of the disclosure comprises at least one (e.g., 1, 2, 3, 4, 5, 6, 7, 8, 9, 10, or more) exNA internucleotide linkage of Formula IIIa. In certain embodiments, the oligonucleotide of the disclosure comprises at least one (e.g., 1, 2, 3, 4, 5, 6, 7, 8, 9, 10, or more) exNA internucleotide linkage of Formula III. In certain embodiments, the oligonucleotide of the disclosure comprises at least one (e.g., 1, 2, 3, 4, 5, 6, 7, 8, 9, 10, or more) exNA internucleotide linkage of Formula IVa. In certain embodiments, the oligonucleotide of the disclosure comprises at least one (e.g., 1, 2, 3, 4, 5, 6, 7, 8, 9, 10, or more) exNA internucleotide linkage of Formula IV. In certain embodiments, the oligonucleotide of the disclosure comprises at least one (e.g., 1, 2, 3, 4, 5, 6, 7, 8, 9, 10, or more) exNA internucleotide linkage of Formula Va. In certain embodiments, the oligonucleotide of the disclosure comprises at least one (e.g., 1, 2, 3, 4, 5, 6, 7, 8, 9, 10, or more) exNA internucleotide linkage of Formula V. In certain embodiments, the oligonucleotide of the disclosure comprises at least one (e.g., 1, 2, 3, 4, 5, 6, 7, 8, 9, 10, or more) exNA internucleotide linkage of Formula VIa. In certain embodiments, the oligonucleotide of the disclosure comprises at least one (e.g., 1, 2, 3, 4, 5, 6, 7, 8, 9, 10, or more) exNA internucleotide linkage of Formula VI.

### Methods of Introducing Nucleic Acids, Vectors and Host Cells

RNA silencing agents of the disclosure may be directly introduced into the cell (e.g., a kidney cell) (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The RNA silencing agents of the disclosure can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or other-wise increase inhibition of the target gene.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus, the RNA may be introduced along with components that perform one or more of the following activities: enhance RNA uptake by the cell, inhibit annealing of single strands, stabilize the single strands, or other-wise increase inhibition of the target gene.

RNA may be directly introduced into the cell (i.e., intracellularly); or introduced extracellularly into a cavity, interstitial space, into the circulation of an organism, introduced orally, or may be introduced by bathing a cell or organism in a solution containing the RNA. Vascular or extravascular circulation, the blood or lymph system, and the cerebrospinal fluid are sites where the RNA may be introduced.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of double stranded RNA material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, Enzyme Linked ImmunoSorbent Assay (ELISA), Western blotting, RadioImmunoAssay (RIA), other immunoassays, and Fluorescence Activated Cell Sorting (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present disclosure. Lower doses of injected material and longer times after administration of RNAi agent may result in inhibition in a smaller fraction of cells (e.g., at least 10%, 20%, 50%, 75%, 90%, or 95% of targeted cells). Quantization of gene expression in a cell may show similar amounts of inhibition at the level of accumulation of target mRNA or translation of target protein. As an example, the efficiency of inhibition may be determined by assessing the amount of gene product in the cell; mRNA may be detected with a hybridization probe having a nucleotide sequence outside the region used for the inhibitory double-stranded RNA, or translated polypeptide may be detected with an antibody raised against the polypeptide sequence of that region.

The RNA may be introduced in an amount which allows delivery of at least one copy per cell. Higher doses (e.g., at least 5, 10, 100, 500 or 1000 copies per cell) of material may yield more effective inhibition; lower doses may also be useful for specific applications.

### Recombinant Adeno-Associated Viruses and Vectors

In certain exemplary embodiments, recombinant adeno-associated viruses (rAAVs) and their associated vectors can be used to deliver one or more siRNAs into cells, e.g., kidney cells. AAV is able to infect many different cell types, although the infection efficiency varies based upon serotype, which is determined by the sequence of the capsid protein. Several native AAV serotypes have been identified, with serotypes 1-9 being the most commonly used for recombinant AAV. AAV-2 is the most well-studied and published serotype. The AAV-DJ system includes serotypes AAV-DJ and AAV-DJ/8. These serotypes were created through DNA shuffling of multiple AAV serotypes to produce AAV with hybrid capsids that have improved transduction efficiencies in vitro (AAV-DJ) and in vivo (AAV-DJ/8) in a variety of cells and tissues.

rAAVs may be delivered to a subject in compositions according to any appropriate methods known in the art. An rAAV can be suspended in a physiologically compatible carrier (i.e., in a composition), and may be administered to a subject, i.e., a host animal, such as a human, mouse, rat, cat, dog, sheep, rabbit, horse, cow, goat, pig, guinea pig, hamster, chicken, turkey, a non-human primate (e.g., Macaque) or the like. In certain embodiments, a host animal is a non-human host animal.

Delivery of one or more rAAVs to a mammalian subject may be performed, for example, by intramuscular injection or by administration into the bloodstream of the mammalian subject. Administration into the bloodstream may be by injection into a vein, an artery, or any other vascular conduit. In certain embodiments, one or more rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer virions into the vasculature of an isolated limb to potentially enhance transduction into muscle cells or tissue.

The compositions of the disclosure may comprise an rAAV alone, or in combination with one or more other viruses (e.g., a second rAAV encoding having one or more different transgenes). In certain embodiments, a composition comprises 1, 2, 3, 4, 5, 6, 7, 8, 9, 10 or more different rAAVs each having one or more different transgenes.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of one or more rAAVs is generally in the range of from about 1 ml to about 100 ml of solution containing from about 109 to 1016 genome copies. In some cases, a dosage between about 1011 to 1012 rAAV genome copies is appropriate. In certain embodiments, 1012 rAAV genome copies is effective to target heart, liver, and pancreas tissues. In some cases, stable transgenic animals are produced by multiple doses of an rAAV.

In some embodiments, rAAV compositions are formulated to reduce aggregation of AAV particles in the composition, particularly where high rAAV concentrations are present (e.g., about 1013 genome copies/mL or more). Methods for reducing aggregation of rAAVs are well known in the art and, include, for example, addition of surfactants, pH adjustment, salt concentration adjustment, etc. (See, e.g., Wright et al. (2005) Molecular Therapy 12:171-178, the contents of which are incorporated herein by reference.)

“Recombinant AAV (rAAV) vectors” comprise, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., siRNA) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are usually about 145 basepairs in length. In certain embodiments, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including mammalian AAV types described further herein.

### Methods of Treatment

In one aspect, the present disclosure provides for both prophylactic and therapeutic methods of treating a subject at risk of (or susceptible to) developing kidney diseases.

“Treatment,” or “treating,” as used herein, is defined as the application or administration of a therapeutic agent (e.g., a RNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the disclosure provides a method for preventing in a subject, a disease or disorder as described above, by administering to the subject a therapeutic agent (e.g., an RNAi agent or vector or transgene encoding same). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the disclosure pertains to methods treating subjects therapeutically, i.e., alter onset of symptoms of the disease or disorder. In an exemplary embodiment, the modulatory method of the disclosure involves contacting a kidney cell with a therapeutic agent (e.g., a RNAi agent or vector or transgene encoding same) that is specific for a target sequence within the gene (e.g., a gene expressed in the kidney), such that sequence specific interference with the gene is achieved. These methods can be performed in vitro (e.g., by culturing the cell with the agent) or, alternatively, in vivo (e.g., by administering the agent to a subject).

### Pharmaceutical Compositions and Methods of Administration

The disclosure pertains to uses of the above-described agents for prophylactic and/or therapeutic treatments as described infra. Accordingly, the modulators (e.g., RNAi agents) of the present disclosure can be incorporated into pharmaceutical compositions suitable for administration. Such compositions typically comprise the nucleic acid molecule or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, and subcutaneous.

The nucleic acid molecules of the disclosure can be inserted into expression constructs, e.g., viral vectors, retroviral vectors, expression cassettes, or plasmid viral vectors, e.g., using methods known in the art, including but not limited to those described in Xia et al., (2002), Supra. Expression constructs can be delivered to a subject by, for example, inhalation, orally, intravenous injection, local administration (see U.S. Pat. No. 5,328,470) or by stereotactic injection (see e.g., Chen et al. (1994), Proc. Natl. Acad. Sci. USA, 91, 3054-3057). The pharmaceutical preparation of the delivery vector can include the vector in an acceptable diluent, or can comprise a slow-release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The nucleic acid molecules of the disclosure can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides. Brummelkamp et al. (2002), Science, 296, 550-553; Lee et al, (2002). supra; Miyagishi and Taira (2002), Nature Biotechnol., 20, 497-500; Paddison et al. (2002), supra; Paul (2002), supra; Sui (2002) supra; Yu et al. (2002), supra.

The expression constructs may be any construct suitable for use in the appropriate expression system and include, but are not limited to retroviral vectors, linear expression cassettes, plasmids and viral or virally-derived vectors, as known in the art. Such expression constructs may include one or more inducible promoters, RNA Pol III promoter systems such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct, Tuschl (2002), Supra.

For example, compositions can include one or more species of a compound of the disclosure and a pharmaceutically acceptable carrier. The pharmaceutical compositions of the present disclosure may be administered by intravenous or subcutaneous injection. or.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following example, which is included for purposes of illustration only and is not intended to be limiting.

### EXAMPLES

### Example 1. Screening of siRNAs Against SLC5A2 Using Dual Glo Luciferase Reporter Assay

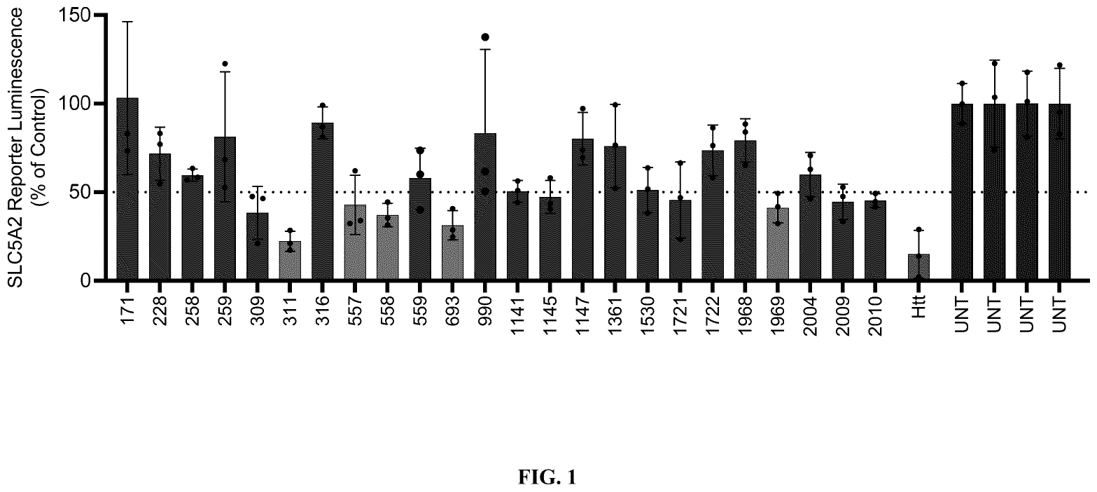

SiRNAs targeting SLC5A2, an exemplary gene target expressed in the kidney, were screened using dual Glo luciferase reporter assay (FIG. 1). To probe the activity of oligonucleotide sequences, target sequences were assayed in vitro. Cells transfected with psiCheck-2 vector (Promega) containing siRNA targeting regions cloned upstream of the luciferase reporter gene were assessed using luminescence measurements. Five siRNA targeting SLC5A2 gene regions 311, 557, 558, 693, and 1969 achieved exceptional silencing and were taken forward for further analysis.

### Example 2. Characterization of Hit siRNA Potency Through 7-Point Dose Response Assays

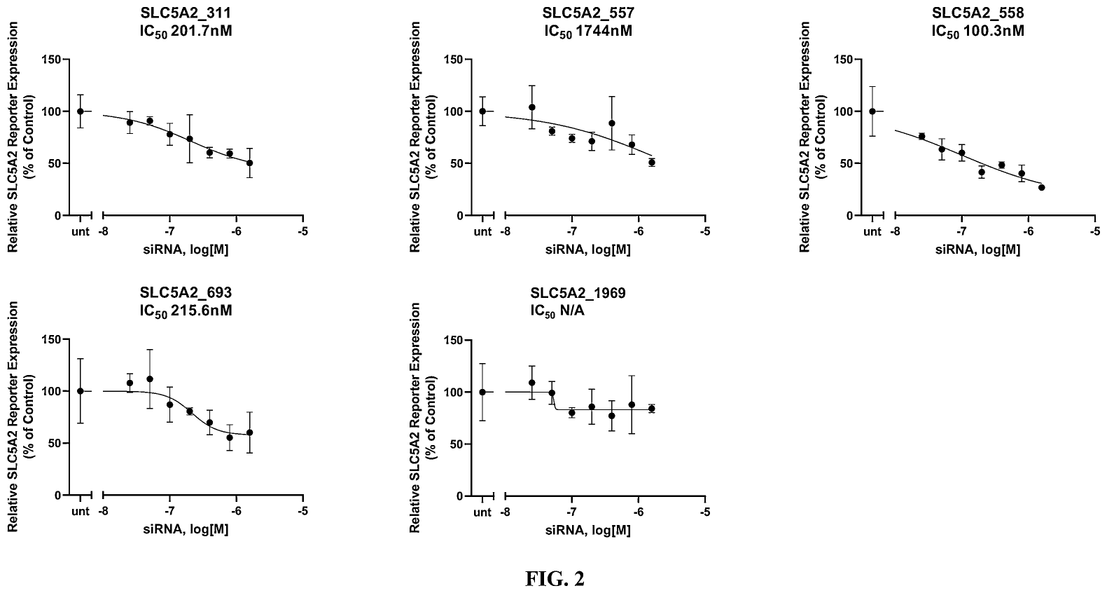

The potency of hit siRNAs was assessed through 7-point dose response assays (FIG. 2). Lead sequences 311, 557, 558, and 693, and 1969 were characterized in vitro. Cells transfected with psiCheck-2 vector (Promega) containing siRNA targeting regions cloned upstream of the luciferase reporter gene were assessed using luminescence measurements.

### Example 3. In Vivo Kidney Silencing

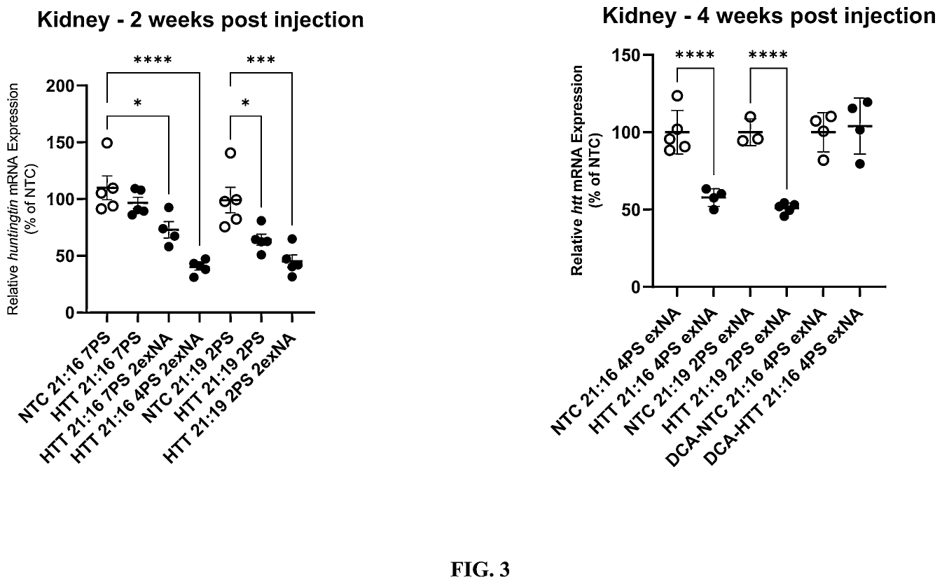

In vivo gene silencing in the kidney is challenging, in part, due to non-specific entrapment of oligonucleotides during filtration. It was hypothesized that the exNA internucleotide modification could be useful in enhancing target mRNA silencing in the kidney. Accordingly, in vivo kidney silencing was assessed (FIG. 3) with a test gene target, HTT. The relative HTT mRNA expression of FVB mice treated sub-cutaneously with siRNA targeting mouse huntingtin (HTT) or non-targeting control (NTC) was measured. At 2-weeks post-administration, silencing was highest with the oligonucleotides containing exNA modifications (see HTT 21:16 7PS 2exNA and HTT 21:16 4PS 2exNA compared against HTT 21:16 7PS). The exNA-containing oligonucleotides in the 21:19 format only silenced HTT slightly better compared to the non-exNA oligonucleotide (HTT 21:19 2PS). This is due to the fact that the 21:19 format siRNA already achieves near maximal silencing of the target and differences in silencing efficacy between differently modified siRNA are more difficult to discern at a 2-week timepoint. The difference between exNA oligonucleotides and non-exNA oligonucleotides is more apparent in the 21:16 siRNA format because it is a less efficient silencing format.

At the 4-week post administration timepoint, oligonucleotides with the exNA modification were found to effectively silence HTT in the kidney. Notably, oligonucleotides without the exNA modification were found to not silence at 4 weeks (data not shown). This is likely due to the fact that the oligonucleotides without the exNA modification are degraded and/or eliminated after the longer timepoint.

Accordingly, oligonucleotides targeting mRNA expressed in the kidney with the exNA internucleotide modification are likely to be effective at silencing said target mRNA in the kidney.

### Example 4. Accumulation and Silencing of siRNA in Various Tissues

The accumulation and silencing of htt targeting siRNA in various tissues were assessed. siRNA targeting sequences are shown in Table 3 (sequence nos. 34497-35501).

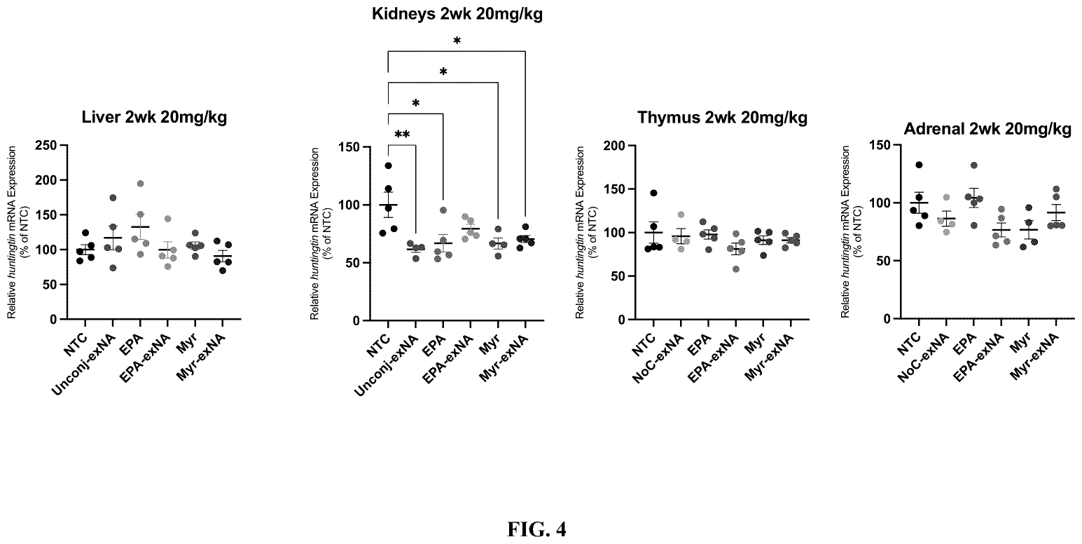

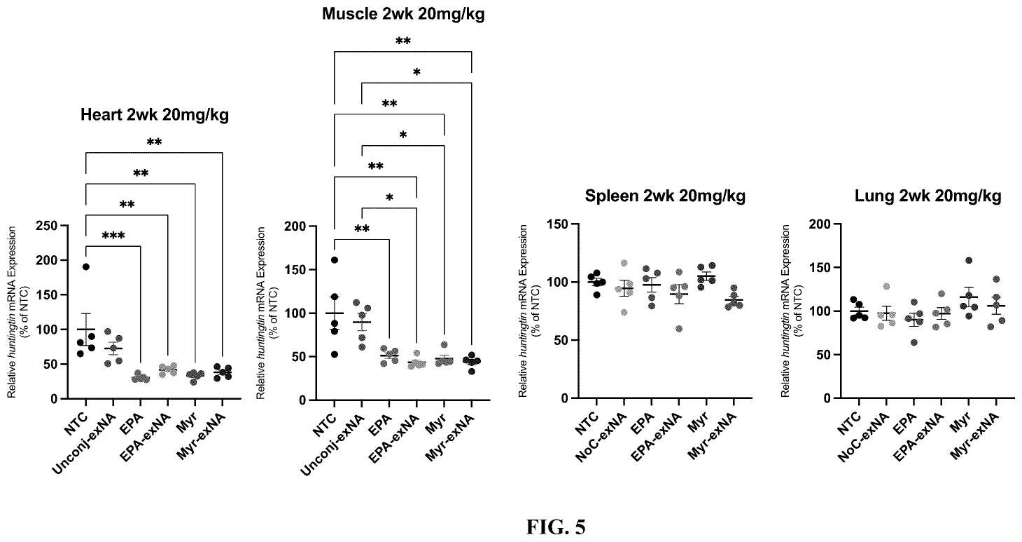

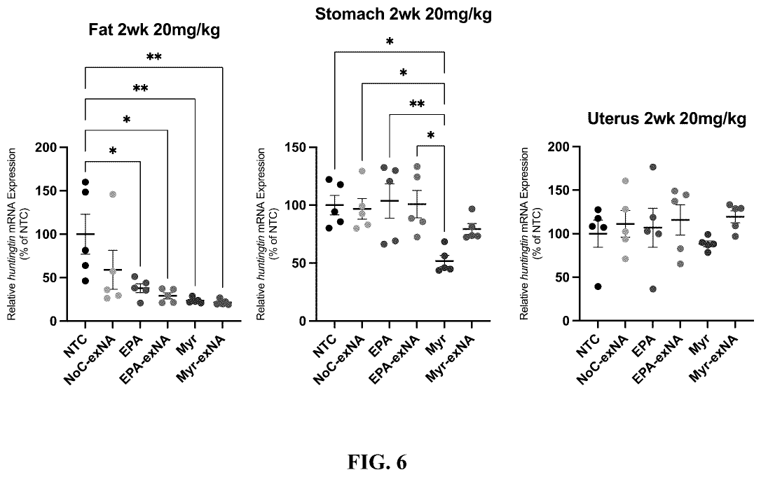

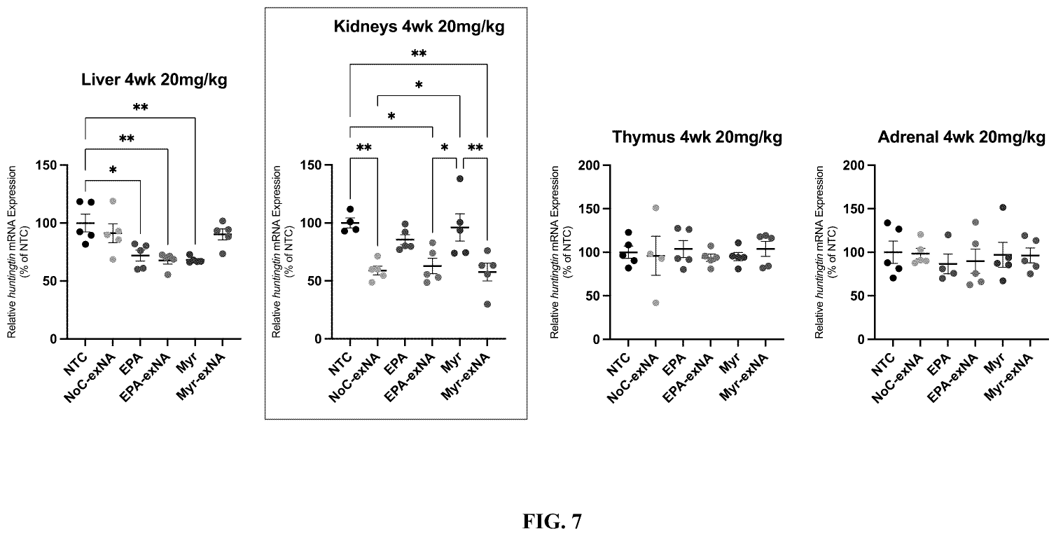

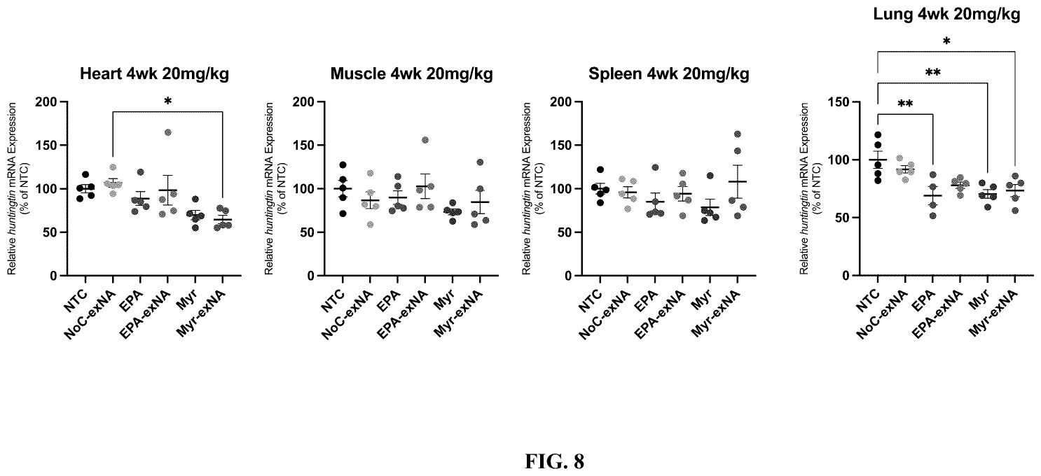

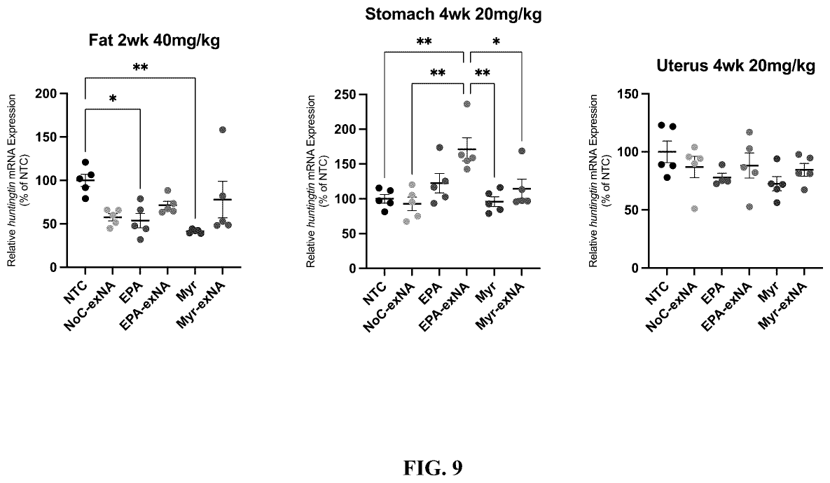

The relative huntingtin mRNA expression in liver, kidney, thymus, adrenal, heart, muscle, spleen, lung, fat, stomach, and uterus tissues of FVB mice treated sub-cutaneously with 20 mg/kg siRNA targeting mouse huntingtin (HTT) or non-targeting control (NTC) 2 weeks (FIGS. 4-6) and 4 weeks (FIGS. 7-9) post injection was measured. Animal experiments were performed in accordance with animal care ethics approval and guidelines of the University of Massachusetts Chan Medical School Institutional Animal Care and Use Committee (IACUC, protocol no. A-2411). 6-8-week-old FVB/N female mice (*22-25 g) obtained from Charles River (The Jackson Laboratory, Bar Harbor, ME) were treated with 40 mg/kg siRNA by intrascapular subcutaneous injection with 150 μL PBS (PBS controls) or with the indicated amount of siRNA (DCA conjugated) suspended in 150 μL PBS. The bulk kidneys were collected, sliced to half and stored in RNAlater (Sigma) at 4° C. overnight. Tissues were lysed in 300 μL Homogenizing Buffer (Affymetrix) containing 0.2 mg/mL proteinase K (Invitrogen) using a QIAGEN TissueLyser II. The lysate was then centrifuged at 1,000×g for 10 min and incubated at 65° C. for 30 minutes. mRNA was quantified using the QuantiGene 2.0 Assay (Affymetrix). Lysates were then analyzed for gene expression using the Quantigene 2.0 branched DNA assay (Invitrogen).

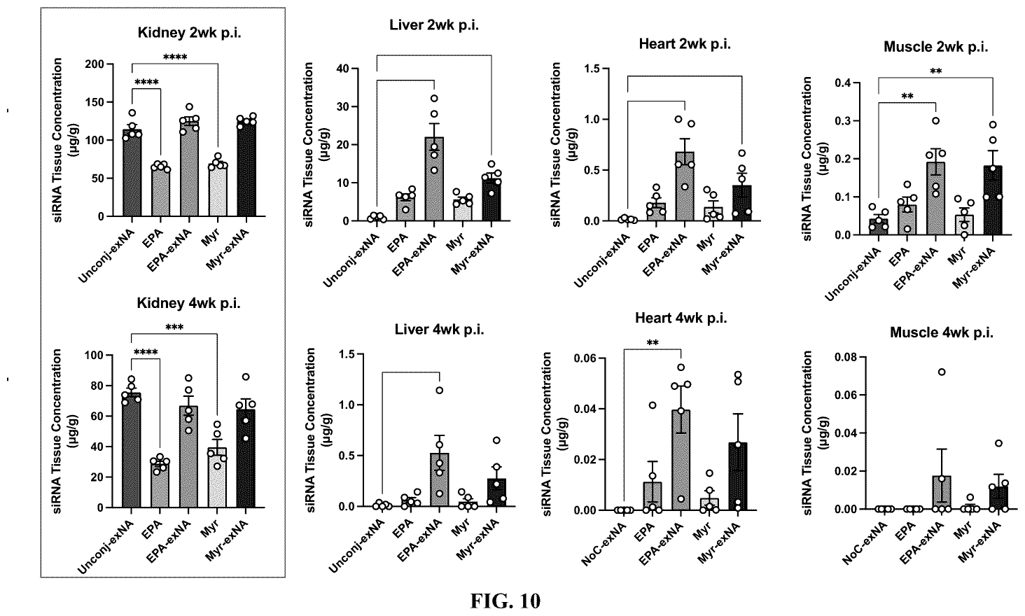

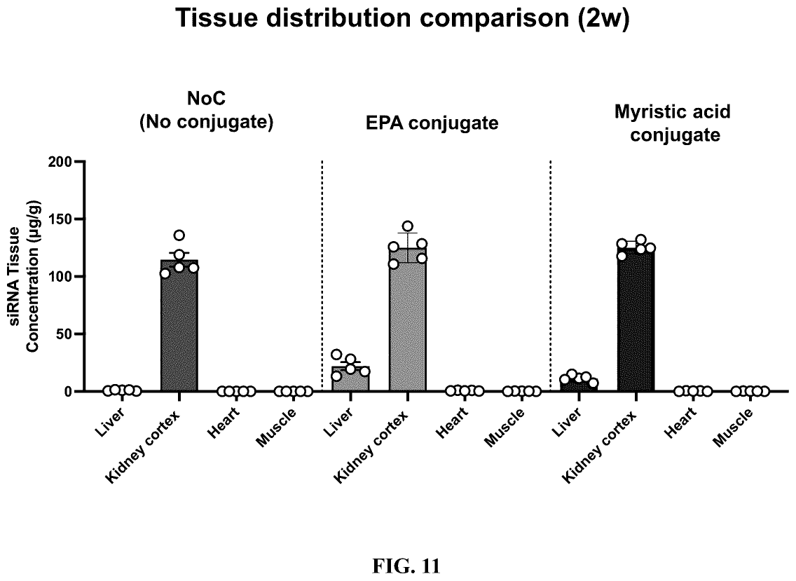

The siRNA concentration in kidney, liver, heart, and muscle tissues of FVB mice treated sub-cutaneously with 20 mg/kg siRNA targeting mouse huntingtin (HTT) or non-targeting control (NTC) 2- and 4-weeks post injection was measured (FIGS. 10-11). Peptide Nucleic Acid (PNA) hybridization assays for tissue siRNA quantification were performed. Quantification of antisense strands in tissues was performed using a PNA hybridization assay. Briefly, tissue lysates from the Quantigene 2.0 Assay described in ‘In vivo mRNA silencing experiments’ were treated with 3 M potassium chloride and pelleted centrifugation at 4,000×g for 15 min to remove all dodecyl sulfate. siRNA in the supernatant were hybridized to an Alexa Fluor 488-labeled PNA probe fully complementary to the antisense strand (PNABio). Samples were analyzed by HPLC (Agilent) over a DNAPac PA100 anion-exchange column (ThermoFisher) with buffer A [50% water, 50% ACN, 25 mM Tris-HCl (pH 8.5) and 1 mM ethylenediaminetetraacetate] and buffer B (800 mM NaClO4 in buffer A). A steep gradient of buffer B (10%-100% within 2.5 min) at a flow rate of 1 mL/min was used for the elution of antisense strand-PNA hybrids. Alexa Fluor 488 fluorescence was monitored, and peaks of the duplex of the antisense strand and PNA probe were integrated to obtain the area under the curve. The final concentrations were ascertained using calibration curves.

### Example 5. sGLT2-558 siRNA Silencing in Kidney

The silencing of siRNA sGLT2-558 in kidney was assessed. The modified SLC5A2 targeting sequences are shown in Table 5.

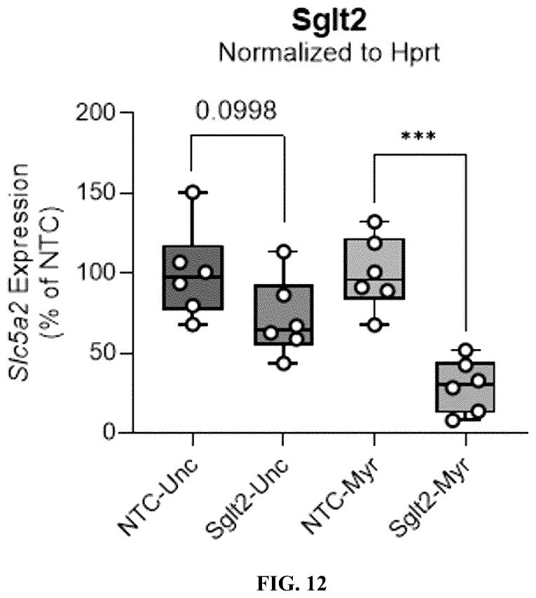

The SLC5A2 expression in kidney tissues of FVB mice treated sub-cutaneously with 20 mg/kg siRNA targeting mouse SLC5A2 or non-targeting control (NTC) was measured (FIG. 12). Animal experiments were performed in accordance with animal care ethics approval and guidelines of the University of Massachusetts Chan Medical School Institutional Animal Care and Use Committee (IACUC, protocol no. A-2411). 6-8-week-old FVB/N female mice (*22-25 g) obtained from Charles River (The Jackson Laboratory, Bar Harbor, ME) were treated with 40 mg/kg siRNA by intrascapular subcutaneous injection with 150 μL PBS (PBS controls) or with the indicated amount of siRNA (DCA conjugated) suspended in 150 μL PBS. The bulk kidneys were collected, sliced to half and stored in RNAlater (Sigma) at 4° C. overnight. Tissues were lysed in 300 μL Homogenizing Buffer (Affymetrix) containing 0.2 mg/mL proteinase K (Invitrogen) using a QIAGEN TissueLyser II. The lysate were then centrifuged at 1,000×g for 10 min and incubated at 65° C. for 30 minutes. mRNA was quantified using the QuantiGene 2.0 Assay (Affymetrix). Lysates were then analyzed for gene expression using the Quantigene 2.0 branched DNA assay (Invitrogen). Unc: No conjugate, Myr: Myristic acid conjugate, and NTC: Non-targeting control.

### Example 6. SLC5A2 Silencing in Kidney and Whole Blood Glucose Level in Mice Injected with siRNA

The SLC5A2 silencing in kidney and whole blood glucose in siRNA injected mice were assessed. The modified SLC5A2 targeting sequences are shown in Table 5.

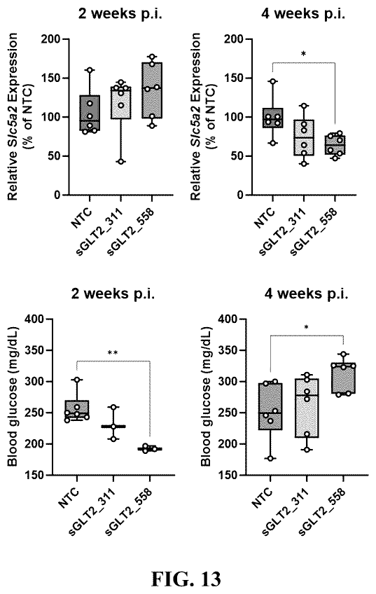

The SLC5A2 expression in kidney tissue and glucose level of FVB mice treated sub-cutaneously with 2×20 mg/kg siRNA targeting mouse SLC5A2 or non-targeting control (NTC) 2- and 4-weeks post injection were assessed (FIG. 13). Animal experiments were performed in accordance with animal care ethics approval and guidelines of the University of Massachusetts Chan Medical School Institutional Animal Care and Use Committee (IACUC, protocol no. A-2411). 6-8-week-old FVB/N female mice (*22-25 g) obtained from Charles River (The Jackson Laboratory, Bar Harbor, ME) were treated with 20 mg/kg (×2 injections) siRNA by intrascapular subcutaneous injection with the indicated amount of siRNA suspended in 150 μL PBS. The bulk kidneys were collected, sliced to half and stored in RNAlater (Sigma) at 4° C. overnight. Tissues were lysed in 300 μL Homogenizing Buffer (Affymetrix) containing 0.2 mg/mL proteinase K (Invitrogen) using a QIAGEN TissueLyser II. The lysate were then centrifuged at 1,000×g for 10 min and incubated at 65° C. for 30 minutes. mRNA was quantified using the QuantiGene 2.0 Assay (Affymetrix). Lysates were then analyzed for gene expression using the Quantigene 2.0 branched DNA assay (Invitrogen).

For the whole blood glucose assay, terminal whole blood was collected in lithium heparin-coated tubes and run on the Abaxis Chemistry Analyzer using the Preventative Care Cartridges in the Department of Animal Medicine.

### Example 7. Whole Blood Glucose Level in Mice Injected with siRNA

The whole blood glucose level in mice injected with siRNA were assessed. The modified SLC5A2 targeting sequences are shown in Table 5.

The whole blood glucose level of C57BL/6 or db/db mice treated sub-cutaneously with 40 mg/kg siRNA targeting mouse SLC5A2 or non-targeting control (NTC) were assessed. Animal experiments were performed in accordance with animal care ethics approval and guidelines of the University of Massachusetts Chan Medical School Institutional Animal Care and Use Committee (IACUC, protocol no. A-2411). 8-week-old male mice (C57BL/6 or db/db WT for Dock7<m>, Homozygous for Lepr<db>) obtained from Jackson Laboratory were treated with 40 mg/kg siRNA by intrascapular subcutaneous injection with 150 μL of the indicated amount of siRNA (Myr-conjugated) suspended in 150 μL PBS on Days 0 and 28. Nonfasted blood glucose was collected weekly from the mice via cheek bleed using a Contour NextGen blood glucometer (measuring range 20 mg/dL-600 mg/dL).

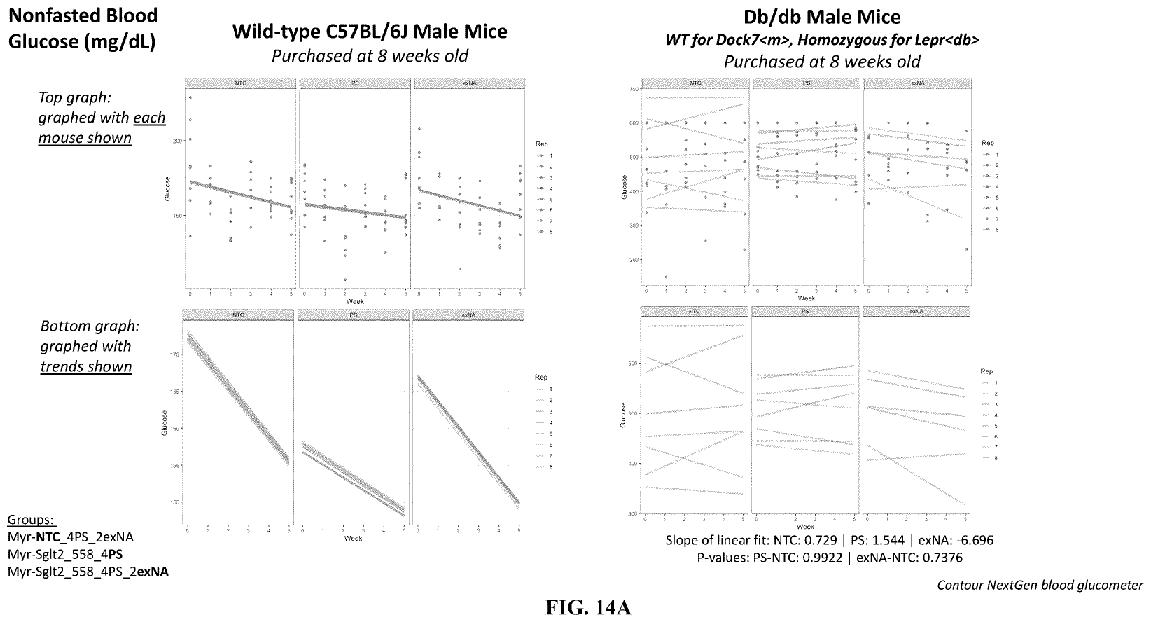

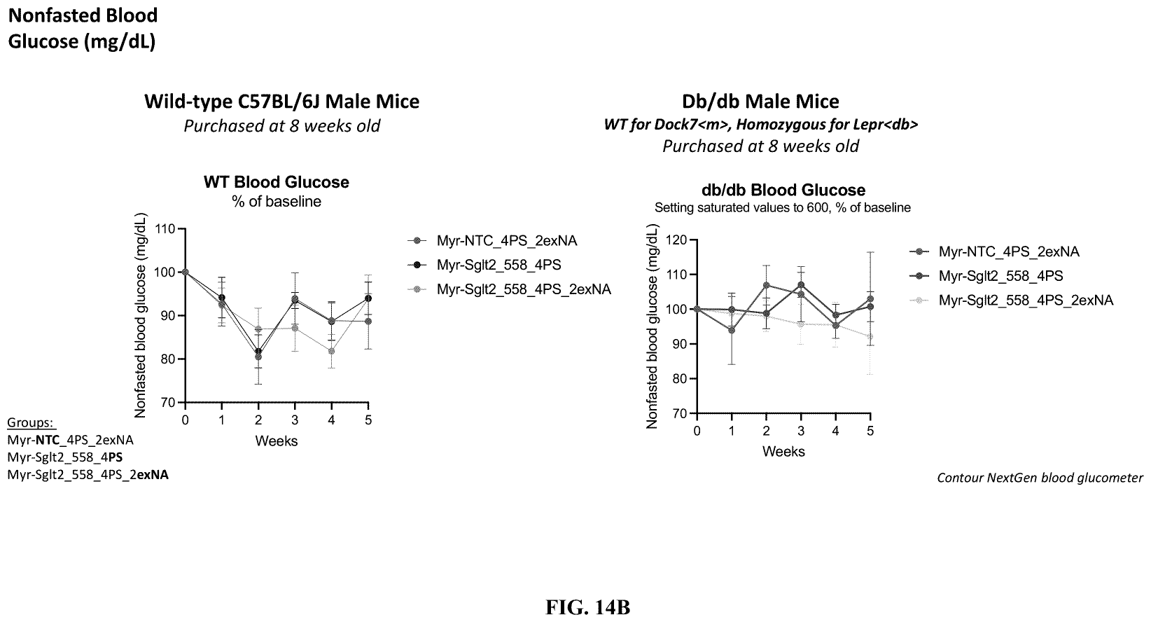

FIG. 14A shows data graphed as glucose level in mg/dL and FIG. 14B shows data graphed as % of each animal's baseline. Blood glucose values were analyzed in R studio using a censored regression model with a right-censoring limit of 600 mg/dL, and plotted lines represent the linear regression based upon other data from animals within the same group (FIG. 14A). Blood glucose values were also analyzed in Prism GraphPad with saturated values set to 600 mg/dL and calculated as a percentage of each animal's baseline (FIG. 14B).

TABLE 1
SLC5A2 targeting sequences.

| Sequence | | SEQ ID | 20 nt Guide | SEQ ID |
|---|---|---|---|---|
| ID | Target Region | Nos: | Sequence | Nos: |
| SLC5A2_ | TCCGTCATCGCGCTTCTGGGCAT | 1 | UAAAUCAUGGUGA | 25 |
| 558 | CACCATGATTTACACGGTGACA | | UGCCCAG | |
| SLC5A2_ | ATGGCAGTGGCCGTGTTCCTC | 2 | GCAUAGAAGCCCCA | 26 |
| 2010 | TGGGGCTTCTATGCCTAAGAC | | GAGGAA | |
| | CAA | | | |
| SLC5A2_ | GAGGTGGGCGGGTATTCGGG | 3 | UAUUUGUCGAAGA | 27 |
| 693 | TCTCTTCGACAAATACCTGGG | | GACCCGA | |
| | AGCA | | | |
| SLC5A2_ | GTTGGGGCCTCTCTCTTCGCC | 4 | CUGCCGAUGUUGCU | 28 |
| 228 | AGCAACATCGGCAGTGGCCA | | GGCGAA | |
| | CTTT | | | |
| SLC5A2_ | CCGAGCTGGGCCCGTGTGGT | 5 | AGGGCAUUGAGGU | 29 |
| 1968 | CAACCTCAATGCCCTGCTCAT | | UGACCAC | |
| | GATG | | | |
| SLC5A2_ | TGAAGCTCATGCCCAACGGT | 6 | CAUGAGUCCGCGCA | 30 |
| 1141 | CTGCGCGGACTCATGCTGGC | | GACCGU | |
| | GGTCA | | | |
| SLC5A2_ | GATGGCAGTGGCCGTGTTCCT | 7 | CAUAGAAGCCCCAG | 31 |
| 2009 | CTGGGGCTTCTATGCCTAAGA | | AGGAAC | |
| | CCA | | | |
| SLC5A2_ | GGCAGTGGCCACTTTGTGGG | 8 | CCAGUCCCUGCCAG | 32 |
| 258 | CCTGGCAGGGACTGGCGCTG | | GCCCAC | |
| | CAAGT | | | |
| SLC5A2_ | CCGTCATCGCGCTTCTGGGCA | 9 | GUAAAUCAUGGUG | 33 |
| 559 | TCACCATGATTTACACGGTGA | | AUGCCCA | |
| | CAG | | | |
| SLC5A2_ | GCTGTTGCTGGATTCGAGTGG | 10 | ACGAAGAGCGCAUU | 34 |
| 309 | AATGCGCTCTTCGTGGTGCTG | | CCACUC | |
| | CTA | | | |
| SLC5A2_ | GCATAGCAAGGAGGAACGGG | 11 | CAGCAUCCAGGUCC | 35 |
| 1721 | AGGACCTGGATGCTGATGAG | | UCCCGU | |
| | CAGCA | | | |
| SLC5A2_ | ATTCCCGAGTTCTCCTTCGGC | 12 | ACACAGCUGCCCGA | 36 |
| 1530 | TCGGGCAGCTGTGTGCAGCC | | GCCGAA | |
| | CTCG | | | |
| SLC5A2_ | GCTCATGCCCAACGGTCTGCG | 13 | CCAGCAUGAGUCCG | 37 |
| 1145 | CGGACTCATGCTGGCGGTCAT | | CGCAGA | |
| | GCT | | | |
| SLC5A2_ | CGAGCTGGGCCCGTGTGGTC | 14 | CAGGGCAUUGAGGU | 38 |
| 1969 | AACCTCAATGCCCTGCTCATG | | UGACCA | |
| | ATGG | | | |
| SLC5A2_ | TCATGCCCAACGGTCTGCGCG | 15 | CGCCAGCAUGAGUC | 39 |
| 1147 | GACTCATGCTGGCGGTCATGC | | CGCGCA | |
| | TGG | | | |
| SLC5A2_ | CATAGCAAGGAGGAACGGGA | 16 | UCAGCAUCCAGGUC | 40 |
| 1722 | GGACCTGGATGCTGATGAGC | | CUCCCG | |
| | AGCAA | | | |
| SLC5A2_ | AAGCTGACGCCCATGTTTCTC | 17 | CCUGGCAUGACCAU | 41 |
| 990 | ATGGTCATGCCAGGCATGATC | | GAGAAA | |
| | AGC | | | |
| SLC5A2_ | GCAGGCGGCACAGGGCGGGC | 18 | UGUAAUCGAAGAGC | 42 |
| 1361 | AGCTCTTCGATTACATCCAGG | | UGCCCG | |
| | CAGT | | | |
| SLC5A2_ | CTCCGTCATCGCGCTTCTGGG | 19 | AAAUCAUGGUGAU | 43 |
| 557 | CATCACCATGATTTACACGGT | | GCCCAGA | |
| | GAC | | | |
| SLC5A2_ | AACAGAGGCACTGTGGGCGG | 20 | CCUGCCAGGAAGUA | 44 |
| 171 | CTACTTCCTGGCAGGACGCA | | GCCGCC | |
| | GCATG | | | |
| SLC5A2_ | CTGGATTCGAGTGGAATGCGC | 21 | CAGCACCACGAAGA | 45 |
| 316 | TCTTCGTGGTGCTGCTACTGG | | GCGCAU | |
| | GCT | | | |
| SLC5A2_ | TGTTGCTGGATTCGAGTGGAA | 22 | CCACGAAGAGCGCA | 46 |
| 311 | TGCGCTCTTCGTGGTGCTGCT | | UUCCAC | |
| | ACT | | | |
| SLC5A2_ | GCAGTGGCCACTTTGTGGGC | 23 | GCCAGUCCCUGCCA | 47 |
| 259 | CTGGCAGGGACTGGCGCTGC | | GGCCCA | |
| | AAGTG | | | |
| SLC5A2_ | CTCATGATGGCAGTGGCCGTG | 24 | AAGCCCCAGAGGAA | 48 |
| 2004 | TTCCTCTGGGGCTTCTATGCC | | CACGGC | |
| | TAA | | | |

TABLE 2
21/16 RNA with UU tail for exNA incorporation.

| | | SEQ ID | | SEQ ID |
|---|---|---|---|---|
| Sequence ID | Guide Sequence | Nos: | Passenger sequence | Nos: |
| SLC5A2_558 | UAAAUCAUGGUGAUGCC | 49 | GCAUCACCAUGAUUUA | 73 |
| | CAUU | | | |
| SLC5A2_2010 | UCAUAGAAGCCCCAGA | 50 | UCUGGGGCUUCUAUGA | 74 |
| | GGAUU | | | |
| SLC5A2_693 | UAUUUGUCGAAGAGAC | 51 | GUCUCUUCGACAAAUA | 75 |
| | CCGUU | | | |
| SLC5A2_228 | UUGCCGAUGUUGCUGG | 52 | CCAGCAACAUCGGCAA | 76 |
| | CGAUU | | | |
| SLC5A2_1968 | UGGGCAUUGAGGUUGA | 53 | UCAACCUCAAUGCCCA | 77 |
| | CCAUU | | | |
| SLC5A2_1141 | UAUGAGUCCGCGCAGA | 54 | UCUGCGCGGACUCAUA | 78 |
| | CCGUU | | | |
| SLC5A2_2009 | UAUAGAAGCCCCAGAG | 55 | CUCUGGGGCUUCUAUA | 79 |
| | GAAUU | | | |
| SLC5A2_258 | UCAGUCCCUGCCAGGC | 56 | GCCUGGCAGGGACUGA | 80 |
| | CCAUU | | | |
| SLC5A2_559 | UUAAAUCAUGGUGAUG | 57 | CAUCACCAUGAUUUAA | 81 |
| | CCCUU | | | |
| SLC5A2_309 | UCGAAGAGCGCAUUCC | 58 | GGAAUGCGCUCUUCGA | 82 |
| | ACUUU | | | |
| SLC5A2_1721 | UAGCAUCCAGGUCCUC | 59 | GAGGACCUGGAUGCUA | 83 |
| | CCGUU | | | |
| SLC5A2_1530 | UCACAGCUGCCCGAGC | 60 | GCUCGGGCAGCUGUGA | 84 |
| | CGAUU | | | |
| SLC5A2_1145 | UCAGCAUGAGUCCGCG | 61 | CGCGGACUCAUGCUGA | 85 |
| | CAGUU | | | |
| SLC5A2_1969 | UAGGGCAUUGAGGUUG | 62 | CAACCUCAAUGCCCUA | 86 |
| | ACCUU | | | |
| SLC5A2_1147 | UGCCAGCAUGAGUCCG | 63 | CGGACUCAUGCUGGCA | 87 |
| | CGCUU | | | |
| SLC5A2_1722 | UCAGCAUCCAGGUCCU | 64 | AGGACCUGGAUGCUGA | 88 |
| | CCCUU | | | |
| SLC5A2_990 | UCUGGCAUGACCAUGA | 65 | UCAUGGUCAUGCCAGA | 89 |
| | GAAUU | | | |
| SLC5A2_1361 | UGUAAUCGAAGAGCUG | 66 | CAGCUCUUCGAUUACA | 90 |
| | CCCUU | | | |
| SLC5A2_557 | UAAUCAUGGUGAUGCC | 67 | GGCAUCACCAUGAUUA | 91 |
| | CAGUU | | | |
| SLC5A2_171 | UCUGCCAGGAAGUAGC | 68 | GCUACUUCCUGGCAGA | 92 |
| | CGCUU | | | |
| SLC5A2_316 | UAGCACCACGAAGAGC | 69 | GCUCUUCGUGGUGCUA | 93 |
| | GCAUU | | | |
| SLC5A2_311 | UCACGAAGAGCGCAUU | 70 | AAUGCGCUCUUCGUGA | 94 |
| | CCAUU | | | |
| SLC5A2_259 | UCCAGUCCCUGCCAGG | 71 | CCUGGCAGGGACUGGA | 95 |
| | CCCUU | | | |
| SLC5A2_2004 | UAGCCCCAGAGGAACA | 72 | UGUUCCUCUGGGGCUA | 96 |
| | CGGUU | | | |

TABLE 3
Modified HTT mRNA targeting sequences.

| ID | Type | Modified Sequence | SEQ ID Nos: |
|---|---|---|---|
| HTT 21:16 | Guide | V(mU)#(fU)#(mA)(fA)(fU)(fC)(mU)(fC)(mU)(fU) | 97 |
| 7PS 2exNA | | (mU)(fA)(mC)(fU)#(mG)#(fA)#(mU)#(mA)#(mU)# | |
| | | (ex-mU)#(ex-fU) | |
| | Passenger | (mA)#(mU)#(mA)(mU)(mC)(mA)(fG)(mU)(mA)(mA) | 8 |
| | | (fA)(mG)(fA)(mG)#(fA)#(mU) | |
| HTT 21:16 | Guide | V(mU)#(fU)#(mA)(fA)(fU)(fC)(mU)(fC)(mU)(fU) | 99 |
| 4PS 2exNA | | (mU)(fA)(mC)(fU)(mG)(fA)(mU)#(mA)#(mU)# | |
| | | (ex-mU)#(ex-fU) | |
| | Passenger | (mA)#(mU)#(mA)(mU)(mC)(mA)(fG)(mU)(mA)(mA) | 98 |
| | | (fA)(mG)(fA)(mG)#(fA)#(mU) | |
| HTT 21:19 | Guide | V(mU)#(fU)#(mA)(fA)(fU)(fC)(mU)(fC)(mU)(fU) | 100 |
| 2PS 2exNA | | (mU)(fA)(mC)(fU)(mG)(fA)(mU)(mA)(mU)# | |
| | | (ex-mU)#(ex-fU) | |
| | Passenger | V(mU)#(fA)#(mU)(fC)(fG)(fG)(mG)(fA)(mA)(fG) | 101 |
| | | (mC)(fU)(mU)(fU)(mG)(fU)(mC)#(mA)#(mG)# | |
| | | (ex-mU)#(ex-fU) | |
| Htt- | Guide | V(mU)#(fU)#(mA)(fA)(fU)(fC)(mU)(fC)(mU)(fU) | 102 |
| AS_Control | | (mU)(fA)(mC)(fU)(mG)(fA)(mU)#(mA)#(mU)#(mU)# | |
| 34497 | | (fU) | |
| Htt- | Guide | V(mU)#(fU)#(mA)(fA)(fU)(fC)(mU)(fC)(mU)(fU) | 103 |
| AS_exNA | | (mU)(fA)(mC)(fU)(mG)(fA)(mU)#(mA)#(mU)#(mxU)# | |
| 34498 | | (fxU) | |
| Htt-SS_Noc | Passenger | (mU)#(mC)#(mA)(fG)(mU)(mA)(mA)(fA)(mG)(fA) | 104 |
| 34499 | | (mG)(fA)(mU)(fU)#(mA)#(mA) | |
| Htt-SS_Myr | Passenger | (mU)#(mC)#(mA)(fG)(mU)(mA)(mA)(fA)(mG)(fA) | 105 |
| 34500 | | (mG)(fA)(mU)(fU)#(mA)#(mA)-Myr | |
| Htt-SS_EPA | Passenger | (mU)#(mC)#(mA)(fG)(mU)(mA)(mA)(fA)(mG)(fA) | 106 |
| 34501 | | (mG)(fA)(mU)(fU)#(mA)#(mA)-epa | |

“m” represents a 2′-O-methyl nucleotide modification; “f” represents a 2′-Fluoro nucleotide modification; “#” represents a phosphorothioate internucleotide linkage; ““V” represents a 5′ vinyl phosphate; “ex” represents an exNA-modified internucleotide linkage; “fx” represents a 2′-fluoro-exNA; “mx” represents a 2′-Ome-exNA; “Myr” represents a myristic acid conjugate; and “EPA” represents an eicosapentaenoic acid.

TABLE 4
Modified SLC5A2 mRNA targeting sequences.

| | | | SEQ ID |
|---|---|---|---|
| ID | Name | Antisense Sequence | Nos: |
| 27654 | SLC5A2_558_as_ | P(mU)#(fA)#(mA)(fA)(fU)(fC)(mA)(fU)(mG) | 107 |
| | P3_UU_Tail_21 | (fG)(mU)(fG)(mA)(fU)#(mG)#(fC)#(mC)#(mC)# | |
| | mer | (mA)#(mU)#(fU) | |
| 27655 | SLC5A2_2010_as_ | P(mU)#(fC)#(mA)(fU)(fA)(fG)(mA)(fA)(mG) | 108 |
| | P3_UU_Tail_21 | (fC)(mC)(fC)(mC)(fA)#(mG)#(fA)#(mG)#(mG)# | |
| | mer | (mA)#(mU)#(fU) | |
| 27656 | SLC5A2_693_as_ | P(mU)#(fA)#(mU)(fU)(fU)(fG)(mU)(fC)(mG) | 109 |
| | P3_UU_Tail_21 | (fA)(mA)(fG)(mA)(fG)#(mA)#(fC)#(mC)#(mC)# | |
| | mer | (mG)#(mU)#(fU) | |
| 27657 | SLC5A2_228_as_ | P(mU)#(fU)#(mG)(fC)(fC)(fG)(mA)(fU)(mG) | 110 |
| | P3_UU_Tail_21 | (fU)(mU)(fG)(mC)(fU)#(mG)#(fG)#(mC)#(mG)# | |
| | mer | (mA)#(mU)#(fU) | |
| 27658 | SLC5A2_1968_as_ | P(mU)#(fG)#(mG)(fG)(fC)(fA)(mU)(fU)(mG) | 111 |
| | P3_UU_Tail_21 | (fA)(mG)(fG)(mU)(fU)#(mG)#(fA)#(mC)#(mC)# | |
| | mer | (mA)#(mU)#(fU) | |
| 27659 | SLC5A2_1141_as_ | P(mU)#(fA)#(mU)(fG)(fA)(fG)(mU)(fC)(mC) | 112 |
| | P3_UU_Tail_21 | (fG)(mC)(fG)(mC)(fA)#(mG)#(fA)#(mC)#(mC)# | |
| | mer | (mG)#(mU)#(fU) | |
| 27660 | SLC5A2_2009_as_ | P(mU)#(fA)#(mU)(fA)(fG)(fA)(mA)(fG)(mC) | 113 |
| | P3_UU_Tail_21 | (fC)(mC)(fC)(mA)(fG)#(mA)#(fG)#(mG)#(mA)# | |
| | mer | (mA)#(mU)#(fU) | |
| 27661 | SLC5A2_258_as_ | P(mU)#(fC)#(mA)(fG)(fU)(fC)(mC)(fC)(mU)(fG) | 114 |
| | P3_UU_Tail_21 | (mC)(fC)(mA)(fG)#(mG)#(fC)#(mC)#(mC)# | |
| | mer | (mA)#(mU)#(fU) | |
| 27662 | SLC5A2_559_as_ | P(mU)#(fU)#(mA)(fA)(fA)(fU)(mC)(fA)(mU) | 115 |
| | P3_UU_Tail_21 | (fG)(mG)(fU)(mG)(fA)#(mU)#(fG)#(mC)#(mC)# | |
| | mer | (mC)#(mU)#(fU) | |
| 27663 | SLC5A2_309_as_ | P(mU)#(fC)#(mG)(fA)(fA)(fG)(mA)(fG)(mC) | 116 |
| | P3_UU_Tail_21 | (fG)(mC)(fA)(mU)(fU)#(mC)#(fC)#(mA)#(mC)# | |
| | mer | (mU)#(mU)#(fU) | |
| 27664 | SLC5A2_1721_as_ | P(mU)#(fA)#(mG)(fC)(fA)(fU)(mC)(fC)(mA) | 117 |
| | P3_UU_Tail_21 | (fG)(mG)(fU)(mC)(fC)#(mU)#(fC)#(mC)#(mC)# | |
| | mer | (mG)#(mU)#(fU) | |
| 27665 | SLC5A2_1530_as_ | P(mU)#(fC)#(mA)(fC)(fA)(fG)(mC)(fU)(mG)(fC) | 118 |
| | P3_UU_Tail_21 | (mC)(fC)(mG)(fA)#(mG)#(fC)#(mC)#(mG)#(mA) | |
| | mer | #(mU)#(fU) | |
| 27666 | SLC5A2_1145_as_ | P(mU)#(fC)#(mA)(fG)(fC)(fA)(mU)(fG)(mA) | 119 |
| | P3_UU_Tail_21 | (fG)(mU)(fC)(mC)(fG)#(mC)#(fG)#(mC)#(mA)# | |
| | mer | (mG)#(mU)#(fU) | |
| 27667 | SLC5A2_1969_as_ | P(mU)#(fA)#(mG)(fG)(fG)(fC)(mA)(fU)(mU) | 120 |
| | P3_UU_Tail_21 | (fG)(mA)(fG)(mG)(fU)#(mU)#(fG)#(mA)#(mC)# | |
| | mer | (mC)#(mU)#(fU) | |
| 27668 | SLC5A2_1147_as_ | P(mU)#(fG)#(mC)(fC)(fA)(fG)(mC)(fA)(mU) | 121 |
| | P3_UU_Tail_21 | G(f)(mA)(fG)(mU)(fC)#(mC)#(fG)#(mC)#(mG)# | |
| | mer | (mC)#(mU)#(fU) | |
| 27669 | SLC5A_1722_as_ | P(mU)#(fC)#(mA)(fG)(fC)(fA)(mU)(fC)(mC)(fA) | 122 |
| | P3_UU_Tail_21 | (mG)(fG)(mU)(fC)#(mC)#(fU)#(mC)#(mC)# | |
| | mer | (mC)#(mU)#(fU) | |
| 27670 | SLC5A2_990_as_ | P(mU)#(fC)#(mU)(fG)(fG)(fC)(mA)(fU)(mG) | 123 |
| | P3_UU_Tail_21 | (fA)(mC)(fC)(mA)(fU)#(mG)#(fA)#(mG)#(mA)# | |
| | mer | (mA)#(mU)#(fU) | |
| 27671 | SLC5A2_1361_as_ | P(mU)#(fG)#(mU)(fA)(fA)(fU)(mC)(fG)(mA) | 124 |
| | P3_UU_Tail_21 | (fA)(mG)(fA)(mG)(fC)#(mU)#(fG)#(mC)#(mC)# | |
| | mer | (mC)#(mU)#(fU) | |
| 27672 | SLC5A2_557_as_ | P(mU)#(fA)#(mA)(fU)(fC)(fA)(mU)(fG)(mG) | 125 |
| | P3_UU_Tail_21 | (fU)(mG)(fA)(mU)(fG)#(mC)#(fC)#(mC)#(mA)# | |
| | mer | (mG)#(mU)#(fU) | |
| 27673 | SLC5A2_171_as_ | P(mU)#(fC)#(mU)(fG)(fC)(fC)(mA)(fG)(mG) | 126 |
| | P3_UU_Tail_21 | (fA)(mA)(fG)(mU)(fA)#(mG)#(fC)#(mC)#(mG)# | |
| | rme | (mC)#(mU)#(fU) | |
| 27674 | SLC5A2_316_as_ | P(mU)#(fA)#(mG)(fC)(fA)(fC)(mC)(fA)(mC)(fG) | 127 |
| | P3_UU_Tail_21 | (mA)(fA)(mG)(fA)#(mG)#(fC)#(mG)#(mC)# | |
| | rme | A(m)#(mU)#(fU) | |
| 27675 | SLC5A2_311_as_ | P(mU)#(fC)#(mA)(fC)(fG)(fA)(mA)(fG)(mA) | 128 |
| | P3_UU_Tail_21 | (fG)(mC)(fG)(mC)(fA)#(mU)#(fU)#(mC)#(mC)# | |
| | rme | (mA)#(mU)#(fU) | |
| 27676 | SLC5A2_259_as_ | P(mU)#(fC)#(mC)(fA)(fG)(fU)(mC)(fC)(mC) | 129 |
| | P3_UU_Tail_21 | (fU)(mG)(fC)(mC)(fA)#(mG)#(fG)#(mC)#(mC)# | |
| | mer | (mC)#(mU)#(fU) | |
| 27677 | SLC5A2_2004_as_ | P(mU)#(fA)#(mG)(fC)(fC)(fC)(mC)(fA)(mG)(fA) | 130 |
| | P3_UU_Tail_21 | (mG)(fG)(mA)(fA)#(mC)#(fA)#(mC)#(mG)# | |
| | mer | (mG)#(mU)#(fU) | |

“m” represents a 2′-O-methyl nucleotide modification; “f” represents a 2′-Fluoro nucleotide modification; “#” represents a phosphorothioate internucleotide linkage; “P” represents a 5′ phosphate; “TegChol” represents cholesterol with a TEG linker.

TABLE 5
Modified SLC5A2 mRNA targeting sequences.

| Name | Sequence (5′→3′) | SEQ ID Nos: |
|---|---|---|
| Sglt2-311-as | V(mU)#(fC)#(mA)(fC)(fG)(fA)(mA)(fG)(mA)(fG) | 131 |
| | (mC)(fG)(mC)(fA)(mU)(fU)(mC)#(mC)#(mA)#(mxU)# | |
| | (fxU) | |
| Sglt2-558-as | V(mU)#(fA)#(mA)(fA)(fU)(fC)(mA)(fU)(mG)(fG) | 132 |
| | (mU)(fG)(mA)(fU)(mG)(fC)(mC)#(mC)#(mA)#(mxU)# | |
| | (fxU) | |
| Sglt2-311-s | (mA)#(mA)#(mU)(fG)(mC)(fG)(mC)(fU)(mC)(fU) | 133 |
| | (mU)(mC)(mG)(fU)#(mG)#(mA) | |
| Sglt2-558-s | (mG)#(mC)#(mA)(fU)(mC)(fA)(mC)(fC)(mA)(fU) | 134 |
| | (mG)(mA)(mU)(fU)#(mU)#(mA) | |
| Sglt2-311-s_Myr | (mA)#(mA)#(mU)(fG)(mC)(fG)(mC)(fU)(mC)(fU) | 135 |
| | (mU)(mC)(mG)(fU)#(mG)#(mA)-Myr | |
| Sglt2-558-s_Myr | (mG)#(mC)#(mA)(fU)(mC)(fA)(mC)(fC)(mA)(fU) | 136 |
| | (mG)(mA)(mU)(fU)#(mU)#(mA)-Myr | |

m: 2′-OMe, f: 2′-fluoro, V: 5′-(E)-vinylphosphonate, #: phosphorothioate, fx: 2′-fluoro-exNA, mx: 2′-Ome-exNA, Myr: Myristic acid conjugate.

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A method for delivering an oligonucleotide to a kidney of a subject, or a method for treating a disease, disorder, or injury of the kidney in a subject in need of such treatment, or a method for administering a therapeutically effective amount of an oligonucleotide to the kidney of a subject, the method comprising administering to the subject the oligonucleotide, wherein the oligonucleotide comprises a 5′ end, a 3′ end, at least one exNA intersubunit linkage, and complementarity to a target nucleic acid, and wherein the oligonucleotide is formulated for kidney administration.

2. The method of claim 1, wherein the oligonucleotide comprises the at least one exNA intersubunit linkage between one or more of positions 1 to 4, 1 to 3, and 1 to 2 from the 3′ end of the oligonucleotide.

3. (canceled)

4. The method of claim 1, wherein the nucleotide at one or more of positions 1, 2, 3, and 4 is U.

5. (canceled)

6. The method of claim 1, wherein the at least one exNA intersubunit linkage comprises the exNA intersubunit linkage of Formula Ia: 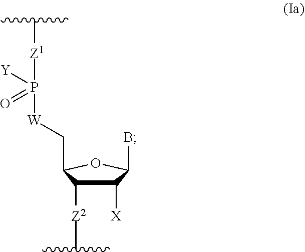
wherein:
B is a base moiety;
W is O or O(CH₂)ₙ¹, wherein n¹ is an integer between 1 and 10;
X is selected from the group consisting of H, OH, OR¹, R¹, F, Cl, Br, I, SH, SR¹, NH₂, NHR¹, NR¹₂, and COOR¹;
Y is selected from the group consisting of O⁻, OH, OR², NH⁻, NH₂, NR²₂, BH₃, S⁻, R², and SH;
Z¹ is O or O(CH₂)ₙ², wherein n² is an integer between 1 and 10; and
Z² is O or O(CH₂)ₙ³, wherein n³ is an integer between 1 and 10,
wherein:
R¹ is a substituted or unsubstituted C₁-C₆ alkyl, alkenyl, alkynyl, or aryl, or mixtures thereof;
R² is a substituted or unsubstituted C₁-C₆ alkyl, alkenyl, alkynyl, or aryl, or mixtures thereof; and
Z₁ is O(CH₂)ₙ² and W is O, or Z₁ is O and W is O(CH₂)ₙ¹, or Z₁ is O(CH₂)ₙ² and W is O(CH₂)ₙ¹.

7. The method of claim 6, wherein:
Z¹ is O(CH₂)ₙ², n² is 1, W is O, and Y is O⁻;
Z¹ is O, W is O(CH₂)ₙ¹, n¹ is 1, and Y is O⁻;
Z¹ is O(CH₂)ₙ², n² is 1, W is O, and Y is S⁻;
Z¹ is O(CH₂)ₙ², n² is 1, W is O(CH₂)ₙ¹, and Y is O⁻; or
Z¹ is O(CH₂)ₙ², n² is 1, W is O(CH₂)ₙ¹, and Y is S⁻.

8-14. (canceled)

15. The method of claim 1, wherein the oligonucleotide is an antisense oligonucleotide (ASO) or a double stranded RNA (dsRNA) moiety, wherein the dsRNA moiety comprises a sense strand and an antisense strand.

16. The method of claim 15, wherein the antisense strand comprises about 15 nucleotides to about 25 nucleotides in length, or is 20 nucleotides in length, 21 nucleotides in length, or 22 nucleotides in length.

17. The method of claim 15, wherein the sense strand comprises about 15 nucleotides to about 25 nucleotides in length, or is 15 nucleotides in length, 16 nucleotides in length, 18 nucleotides in length, 19 nucleotides in length, or 20 nucleotides in length.

18-24. (canceled)

25. The method of claim 15, wherein the dsRNA comprises at least one modified nucleotide, optionally wherein the at least one modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

26. (canceled)

27. The method of claim 15, wherein the dsRNA comprises at least one modified internucleotide linkage other than exNA, optionally wherein the at least one modified internucleotide linkage comprises a phosphorothioate internucleotide linkage.

28-35. (canceled)

36. The method of claim 15, wherein a functional moiety is linked to the 5′ end and/or the 3′ end of the antisense strand or the sense strand.

37-38. (canceled)

39. The method of claim 36, wherein the functional moiety comprises:
an N-acetylgalactosamine (GalNAc) moiety;
a hydrophobic moiety, optionally wherein the hydrophobic moiety is selected from the group consisting of fatty acids, steroids, secosteroids, lipids, gangliosides, nucleoside analogs, endocannabinoids, vitamins, and a mixture thereof, optionally wherein the steroid is selected from the group consisting of cholesterol and lithocholic acid (LCA), and optionally wherein the fatty acid is selected from the group consisting of eicosapentaenoic acid (EPA), docosahexaenoic acid (DHA), and docosanoic acid (DCA); or
a lipophilic moiety, optionally wherein the lipophilic moiety is selected from the group consisting of cholesterols, vitamin E, vitamin K, vitamin A, folic acids, cationic dyes, and a mixture thereof, optionally wherein the lipophilic moiety is selected from the group consisting of cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, phenoxazine, and a mixture thereof.

40-46. (canceled)

47. The method of claim 36, wherein the functional moiety is linked to the antisense strand and/or the sense strand by a linker.

48. The method of claim 47, wherein the linker is a cleavable linker, optionally wherein the cleavable linker comprises a phosphodiester linkage, a disulfide linkage, an acid-labile linkage, a photocleavable linkage, or a dTdT dinucleotide with phosphodiester internucleotide linkages, optionally wherein the acid-labile linkage comprises a β-thiopropionate linkage or a carboxydimethylmaleic anhydride (CDM) linkage.

49-53. (canceled)

54. The method of claim 1, wherein the oligonucleotide is administered to the subject by intravenous (IV) injection, subcutaneous (SQ) injection, or a combination thereof.

55. The method of claim 1, wherein the oligonucleotide is a formulation comprising about 0.1, 0.5, 1.0, 5.0, 10.0, 15.0, or 20.0 mg/kg of body weight of the oligonucleotide.

56-59. (canceled)

60. The method of claim 1, wherein:
the disease, disorder, or injury of the kidney is selected from the group consisting of Chronic Kidney Disease (CKD), Fabry disease, cystinosis, glomerulonephritis, IgA nephropathy, lupus nephritis, atypical hemolytic uremic syndrome (aHUS), Polycystic kidney disease (PKD), and a combination thereof; or
the disease, disorder, or injury of the kidney is associated with a solute carrier family 5 member 2 (SLC5A2) gene.

61. (canceled)

62. The method of claim 60, wherein:
the oligonucleotide inhibits the expression of the SLC5A2 gene; and/or
the target nucleic acid is a sequence of any one of target region sequences of Table 1.

63-64. (canceled)

65. A method for inhibiting the expression of a solute carrier family 5 member 2 (SLC5A2) gene in a cell, the method comprising:
(a) introducing into the cell an oligonucleotide comprising an oligonucleotide comprising a 5′ end, a 3′ end, at least one exNA intersubunit linkage, and complementarity to a target nucleic acid; and
(b) maintaining the cell produced in step (a) for a time sufficient to obtain degradation of a mRNA transcript of the SLC5A2 gene, thereby inhibiting expression of the SLC5A2 gene in the cell.

66. (canceled)

67. A pharmaceutical composition for treating a disease, disorder, or injury of the kidney in a patient in need of such treatment, comprising:
an oligonucleotide comprising a 5′ end, a 3′ end, at least one exNA intersubunit linkage, and complementarity to a target nucleic acid; and
a pharmaceutically acceptable carrier,
wherein the pharmaceutical composition is formulated for kidney administration.
